(19) United States
(12) Patent Application Publication
(10) Pub. No.: US 2020/0143903 A1
(43) Pub. Date: May 7, 2020

(54) METHODS FOR IDENTIFYING COMPOUNDS

(71) Applicant: X-Chem, Inc., Waltham, MA (US)

(72) Inventors: Eric Alan SIGEL, Belmont, MA (US); Ling XUE, Cambridge, MA (US); Christopher James MULHERN, Wayland, MA (US); Dennis Joseph MOCCIA, Amesbury, MA (US)

(21) Appl. No.: 16/606,325

(22) PCT Filed: Apr. 18, 2018
(86) PCT No.: PCT/US2018/028050

§ 371 Date: Oct. 18, 2019

Related U.S. Application Data
(60) Provisional application No. 62/486,692, filed on Apr. 18, 2017.

(51) Int. Cl. G16B 15/30; G16C 60/00

(52) U.S. Cl. CPC G16B 15/30; G16C 60/00

(57) ABSTRACT

The present disclosure provides virtual screening methods utilizing data sets from nucleotide-encoded libraries (e.g., DNA-encoded libraries). These methods allow for high confidence predictions of binding interactions between candidate compounds and proteins of interest useful for the development of therapeutics.

### BACKGROUND

Virtual screening methods are capable of expanding the available screening options for a given target and may increase the likelihood of successful optimization. Virtual screening can be a fast and inexpensive method to identify multiple scaffolds to be used as starting points for optimization. Virtual screening is generally limited in capability by the size of the experimentally determined data set used as it relies on comparison to known experimental data to produce the virtual data. Thus, there is a need for methods which combine robust computational methods with extremely large data sets to produce sufficient confidence in the computational predictions to replace traditional high throughput screening methods.

### SUMMARY OF THE INVENTION

The present disclosure provides methods for identifying compounds useful as therapeutic agents and/or useful as starting points for optimization in the development of therapeutic agents. These methods combine computational methods useful for predicting binding between compounds and proteins with large data sets of experimental data derived using nucleotide-encoded libraries (e.g., DNA-encoded libraries). The combination of data generated with nucleotide-encoded libraries and computational methods allows for high confidence predictions of binding interactions between candidate compounds and proteins of interest.

Accordingly, in one aspect, this disclosure provides a method comprising the steps of: (a) providing a plurality of binding interaction findings (e.g., at least 250,000 findings) for a target protein in a physical computing device having a representation of a set of candidate compounds (e.g., small molecule compounds), wherein at least 50% (e.g., at least 60%, at least 70%, at least 80%, at least 90%, at least 95%, at least 99%) of the binding interaction findings in the plurality are representative of a binding interaction between the target protein and a compound comprising a nucleotide tag encoding the identity of the compound (e.g., a member of a DNA-encoded library); (b) using the computing device to generate estimated binding interactions of the candidate compounds using the plurality of binding interaction findings; and (c) outputting a candidate compound list capable of being displayed and ranked by highest estimated binding interactions.

In some embodiments, the plurality of binding interaction findings comprises at least 250,000 (e.g., at least 500,000, at least one million, at least two million, at least five million, at least ten million, at least twenty five million) binding interaction findings.

In some embodiments, at least 50% of the plurality of binding interaction findings were determined by contacting a plurality (e.g., at least 250,000, at least 500,000, at least one million, at least two million, at least five million, at least ten million) of compounds comprising a nucleotide tag encoding the identity of the compound with a target protein simultaneously (e.g., in the same reaction vessel at the same time). For example, in some embodiments, at least 50% of the binding interactions findings for DNA-encoded library members utilized to generate the estimated binding interactions were determined in a single experiment in a single reaction vessel.

In some embodiments, the method further comprises providing one or more additional pluralities of binding interaction findings for one or more additional target proteins, wherein at least 50% of the binding interaction findings in the one or more additional pluralities are representative of a binding interaction between the additional target protein and a compound from the plurality of binding interaction findings with the target protein of step (a). In some embodiments, the method further comprises providing one or more additional pluralities of binding interaction findings for one or more negative control experiments, wherein at least 50% of the binding interaction findings in the plurality are representative of a negative control of a compound from the plurality of binding interaction findings with the target protein of step (a). In some embodiments, the method further comprises providing one or more additional pluralities of binding interaction findings for one or more control experiments, wherein the plurality of binding interaction findings include binding interaction findings of compound with known binding interactions with the target protein of step (a) (e.g., known inhibitors or natural ligands). In some embodiments, the method includes generating a selectivity score by comparing the binding or estimated binding of a compound or candidate compound to the target protein to the binding or estimated binding of the compound or candidate compound to the one or more additional target proteins and/or negative control. In some embodiments, the candidate compound list is capable of being displayed and ranked by the selectivity score. In some embodiments, the one or more additional target proteins comprise a mutant of the target protein.

In some embodiments, the estimated binding interactions are generated using chemical structure comparisons, e.g., utilizing molecular representations. Molecular representations include, but are not limited to, topological representations based on atoms, features, or functional groups and their connectivity (e.g., fingerprints, connection tables, molecular connectivity, and/or molecular graph representations), electrostatic representations (e.g., surface electronics), geometric representations (e.g., pharmacophores, pharmacophore fingerprints, shape-based fingerprints, and/or 3D molecular coordinates using atoms, features, or functional groups), or quantum-chemical representations. In some embodiments, the estimated binding interactions are generated using topological representations based on atoms, features, or functional groups and their connectivity (e.g., fingerprints, connection tables, molecular connectivity, and/or molecular graph representations). In some embodiments, the estimated binding interactions are generated using electrostatic representations (e.g., surface electronics). In some embodiments, the estimated binding interactions are generated using geometric representations (e.g., pharmacophores, pharmacophore fingerprints, shape-based fingerprints, and/or 3D molecular coordinates using atoms, features, or functional groups). In some embodiments, the estimated binding interactions are generated using quantum-chemical representations. In some embodiments, the estimated binding interactions are generated using chemical fingerprints.

Chemical fingerprints may be used to aggregate structural information of compounds and binding interaction data to identify structural patterns indicative of binding to a target protein. Accordingly, in some embodiments, the method further includes (i) providing a plurality of chemical fingerprints of a plurality of compounds (e.g., chemical fingerprints such as ECFP6, FCFP6, ECFP4, MACCS, or Morgan/Circular Fingerprints with varying number of bits (e.g., 166, 512, 1024)); and (ii) utilizing the plurality of chemical fingerprints in the generation of the estimated binding interactions. In some embodiments, e.g., in training sets, the plurality of chemical fingerprints includes chemical fingerprints of one or more of the compounds comprising a nucleotide tag encoding the identity of the compound, e.g., the chemical fingerprint is a representation of the structure of the compound without the nucleotide tag. In some embodiments, e.g., in prediction sets, the plurality of chemical fingerprints includes chemical fingerprints of one or more of the candidate compounds. In some embodiments, the chemical fingerprints are ECFP6 fingerprints.

In some embodiments, the method further comprises providing one or more property findings (e.g., molecular weight and/or clog P) for the set of candidate compounds. In some embodiments, the one or more property findings are utilized to generate the estimated binding interactions. In some embodiments, the candidate compound list is capable of being displayed and ranked by the one or more property findings.

In some embodiments, the method further comprises transmitting the candidate compound list over the internet or to a display device. In some embodiments, the physical computing device is accessed and operated over the internet.

In some embodiments, the method further comprises generating a believability score for each of the estimated binding interactions of the candidate compounds, wherein the believability score is generated using chemical structure comparisons (e.g., principal component analysis) between the candidate compound and one or more compounds from the plurality of binding interactions for the target protein of step (a). For example, in some embodiments, a believability score is generated by comparing a candidate compound to the chemical space defined by the compounds from the plurality of binding interactions of step (a) by determining a distance, such as a Euclidean distance in dimensions defined by prinicipal component analysis, of the candidate compound to the chemical space. In some embodiments, the candidate compound list is capable of being displayed and ranked by the believability score of the estimated binding interaction for the candidate compound.

In some embodiments, the method further comprises (d) synthesizing one or more of the candidate compounds from the candidate compound list.

In some embodiments, the method further comprises (e) contacting one or more synthesized candidate compounds with the target protein to determine one or more experimental binding interactions.

In an aspect, the disclosure provides a computer readable medium having stored thereon executable instructions for directing a physical computing device to implement a method comprising the steps of:

(a) providing a plurality of binding interaction findings for a target protein in a physical computing device having a representation of a set of candidate compounds, wherein at least 90% of the binding interaction findings in the plurality are representative of a binding interaction between the target protein and a compound comprising a nucleotide tag encoding the identity of the compound;

(b) using the computing device to generate estimated binding interactions of the candidate compounds using the plurality of binding interaction findings; and

(c) outputting a candidate compound list capable of being displayed and ranked by highest estimated binding interactions.

In an aspect, the disclosure provides, a physical computing device having a representation of a set of candidate compounds and programmed with executable instructions for directing the device to implement a method comprising the steps of:

(a) providing a plurality of binding interaction findings for a target protein in a physical computing device having a representation of a set of candidate compounds, wherein at least 90% of the binding interaction findings in the plurality are representative of a binding interaction between the target protein and a compound comprising a nucleotide tag encoding the identity of the compound;

(b) using the computing device to generate estimated binding interactions of the candidate compounds using the plurality of binding interaction findings; and

(c) outputting a candidate compound list capable of being displayed and ranked by highest estimated binding interactions.

### Definitions

A “believability score,” as used herein refers to a calculation that indicates the confidence in an estimated binding interaction for a candidate compound based on the structural similarity between the candidate compound and one or more compounds in the data set utilized to prepare the estimate.

The term “binding interaction,” as used herein refers to association (e.g., non-covalent or covalent) between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

The affinity of a molecule X for its partner Y can generally be represented by the dissociation constant (K_{D}). Affinity can be measured by common methods known in the art, including those described herein. The term “K_{D},” as used herein, is intended to refer to the dissociation equilibrium constant of a particular compound-protein or complex-protein interaction. Typically, the compounds of the invention bind to presenter proteins with a dissociation equilibrium constant (K_{D}) of less than about 10⁻⁶ M, such as less than approximately 10⁻⁷ M, 10⁻⁸ M, 10⁻⁹ M, or 10⁻¹⁰ M or even lower, e.g., when determined by surface plasmon resonance (SPR) technology using the presenter protein as the analyte and the compound as the ligand. In some embodiments, the compounds of the invention bind to target proteins (e.g., a eukaryotic target protein such as a mammalian target protein or a fungal target protein or a prokaryotic target protein such as a bacterial target protein) with a dissociation equilibrium constant (K_{D}) of less than about 10⁻⁶ M, such as less than approximately 10⁻⁷ M, 10⁻⁸ M, 10⁻⁹ M, or 10⁻¹⁰ M or even lower, e.g., when determined by surface plasmon resonance (SPR) technology using the target protein as the analyte and the compound as the ligand.

A “binding interaction finding,” as used herein refers to a binding interaction or lack thereof between a compound and a protein (e.g., a target protein) which has been experimentally determined, e.g., by SPR. For example, in some embodiments, a binding interaction finding refers to the determination that a compound does not interact with a protein (e.g., a target protein).

The term “molecular representations” refers to, for example, topological representations, electrostatic representations, geometric representations, or quantum-chemical representations of compounds. Molecular representations include, for example, chemical fingerprints.

The term “electrostatic representations” refers to a type of molecular representations, including information such as surface electronics.

An “estimated binding interaction,” as used herein refers to a binding interaction which has been predicted using computational analysis. In some embodiments, an estimated binding interaction of a candidate compound with a target protein is generated by comparison of the chemical structure candidate compound to the chemical structure of one or more compounds for which a binding interaction with the target protein has been experimentally determined.

As used herein, the term “chemical fingerprint” refers to machine readable molecular representations of compounds such as a bit string, i.e., a list of binary values (0 or 1), which characterize the two- and/or three-dimensional structure of a molecule. Exemplary methods to generate chemical fingerprints are known in the art including, but not limited to, MACCS, Extended Connectivity Fingerprints (ECFPs), Functional-Class Fingerprints (FCFPs), Morgan/Circular Fingerprints, and Chemical Hashed

Fingerprints.

As used herein, the term “c log P” refers to the calculated partition coefficient of a molecule or portion of a molecule. The partition coefficient is the ratio of concentrations of a compound in a mixture of two immiscible phases at equilibrium (e.g., octanol and water) and measures the hydrophobicity or hydrophilicity of a compound. A variety of methods are available in the art for determining c log P For example, in some embodiments, c log P can be determined using quantitative structure-property relationship algorithms known in the art (e.g., using fragment based prediction methods that predict the log P of a compound by determining the sum of its non-overlapping molecular fragments). Several algorithms for calculating clog P are known in the art including those used by molecular editing software such as CHEMDRAW® Pro, Version 12.0.2.1092 (Cambridgesoft, Cambridge, Mass.) and MARVINSKETCH® (ChemAxon, Budapest, Hungary).

The term “comparable,” as used herein, refers to two or more compounds, entities, situations, sets of conditions, etc that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such compounds, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Many methodologies described herein include a step of “determining.” Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

The term “geometric representations” refers to a type of molecular representation. Geometric representations may include information regarding, for example, pharmacophores, pharmacophore fingerprints, shape-based fingerprints, and/or 3D molecular coordinates using atoms, features, or functional groups.

As used herein, the term “library” refers to a group of 2, 5, 10, 10², 10³, 10⁴, 10⁵, 10⁶, 10⁷, 10⁸, 10⁹, or more different molecules. In some embodiments, at least 10% (e.g., at least 20%, at least 30%, at least 40%, at least 50%, at least 60%, at least 70%, at least 80%, at least 90%, at least 95%, at least 99% or 100%) of the compounds in the library are compounds including a nucleotide tag encoding their identity, such as DNA-encoded compounds.

As used herein, the term “negative control,” refers to an experiment to determine a binding interaction wherein the target protein is absent.

The term “polar surface area” refers to the surface sum over all polar atoms of a molecule or portion of a molecule, including their attached hydrogens. Polar surface area is determined computationally using a program such as CHEMDRAW® Pro, Version 12.0.2.1092 (Cambridgesoft, Cambridge, Mass.).

As used herein, the term “positive control” refers to an experiment to determine a binding interaction wherein the binding affinity of the compound contacted with a target protein is known.

A “property finding,” as used herein refers to a calculated or experimentally determined property (e.g., clog P, polar surface area, molecular weight) of a particular compound.

The term “selective” when used with reference to a compound having an activity, is understood by those skilled in the art to mean that the compound discriminates between potential target entities or states. For example, in some embodiments, a compound is said to bind “selectively” to its target if it binds preferentially with that target in the presence of one or more competing alternative targets. In many embodiments, selective interaction is dependent upon the presence of a particular structural feature of the target entity (e.g., an epitope, a cleft, a binding site). It is to be understood that selectivity need not be absolute. In some embodiments, selectivity may be evaluated relative to that of the binding agent for one or more other potential target entities (e.g., competitors). In some embodiments, selectivity is evaluated relative to that of a reference selective binding agent. In some embodiments, selectivity is evaluated relative to that of a reference non-selective binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

A “selectivity score,” as used herein refers to a calculation of the specificity of a compound for a target protein. In some embodiments, a selectivity score may be calculated by comparison of the binding of the compound to the target protein and the binding of the compound to another protein (e.g., a mutant of the target protein or an unrelated protein). In other embodiments, a selectivity score may be calculated by comparison of the binding of the compound to the target protein and a negative control.

The term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating compound. In some embodiments, a small molecule is biologically active. In some embodiments, a small molecule is detectable (e.g., comprises at least one detectable moiety). In some embodiments, a small molecule is a therapeutic.

Those of ordinary skill in the art, reading the present disclosure, will appreciate that certain small molecule compounds described herein may be provided and/or utilized in any of a variety of forms such as, for example, salt forms, protected forms, pro-drug forms, ester forms, isomeric forms (e.g., optical and/or structural isomers), isotopic forms, etc. In some embodiments, reference to a particular compound may relate to a specific form of that compound. In some embodiments, reference to a particular compound may relate to that compound in any form. In some embodiments, where a compound is one that exists or is found in nature, that compound may be provided and/or utilized in accordance in the present invention in a form different from that in which it exists or is found in nature. Those of ordinary skill in the art will appreciate that a compound preparation including a different level, amount, or ratio of one or more individual forms than a reference preparation or source (e.g., a natural source) of the compound may be considered to be a different form of the compound as described herein. Thus, in some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form; etc.

As used herein, the terms “specific binding” or “specific for” or “specific to” refer to an interaction between a binding agent and a target entity. As will be understood by those of ordinary skill, an interaction is considered to be “specific” if it is favored in the presence of alternative interactions, for example, binding with a K_{D} of less than 10 μM (e.g., less than 5 μM, less than 1 μM, less than 500 nM, less than 200 nM, less than 100 nM, less than 75 nM, less than 50 nM, less than 25 nM, less than 10 nM or 10 nM to 100 nM, 50 nM to 250 nM, 100 nM to 500 nM, 250 nM to 1 μM, 500 nM to 2 μM, 1 μM to 5 μM). In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent.

The term “structural similarity” refers to the similarity of the two or three dimensional arrangement and/or orientation of atoms or moieties relative to one another (for example: distance and/or angles between or among them between an agent of interest and a reference agent) in one or more different compounds.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “does not substantially bind” to a particular protein as used herein can be exhibited, for example, by a molecule or portion of a molecule having a K_{D} for the target of 10⁻⁴ M or greater, alternatively 10⁻⁵ M or greater, alternatively 10⁻⁶ M or greater, alternatively 10⁻⁷ M or greater, alternatively 10⁻⁸ M or greater, alternatively 10⁻⁹ M or greater, alternatively 10⁻¹⁰ M or greater, alternatively 10⁻¹¹ M or greater, alternatively 10⁻¹² M or greater, or a K_{D} in the range of 10⁻⁴ M to 10⁻¹² M or 10⁻⁶ M to 10⁻¹⁰ M or 10⁻⁷ M to 10⁻⁹ M.

The term “target protein” refers to a protein that binds with a small molecule. In some embodiments, the target protein participates in a biological pathway associated with a disease, disorder or condition. In some embodiments, a target protein is a naturally-occurring protein; in some such embodiments, a target protein is naturally found in certain mammalian cells (e.g., a mammalian target protein), fungal cells (e.g., a fungal target protein), bacterial cells (e.g., a bacterial target protein) or plant cells (e.g., a plant target protein). In some embodiments, a target protein is characterized by natural interaction with one or more natural presenter protein/natural small molecule complexes. In some embodiments, a target protein is characterized by natural interactions with a plurality of different natural presenter protein/natural small molecule complexes; in some such embodiments some or all of the complexes utilize the same presenter protein (and different small molecules). Target proteins can be naturally occurring, e.g., wild type. Alternatively, the target protein can vary from the wild type protein but still retain biological function, e.g., as an allelic variant, a splice mutant or a biologically active fragment. Exemplary mammalian target proteins are GTPases, GTPase activating protein, Guanine nucleotide-exchange factor, heat shock proteins, ion channels, coiled-coil proteins, kinases, phosphatases, ubiquitin ligases, transcription factors, chromatin modifier/remodelers, proteins with classical protein-protein interaction domains and motifs, or any other proteins that participate in a biological pathway associated with a disease, disorder or condition.

The term “topological representations” refers to a type of molecular representation which depends on the molecule's topology, and which indicates the position of the individual atoms and the bonded connections between them. Topological representations may be based on atoms, features, or functional groups and their connectivity (e.g., fingerprints, connection tables, molecular connectivity, and/or molecular graph representations). Topological representations may be calculated based on the graphical representation of the molecules.

The term “quantum-chemical representations” refers to a type of molecular representation. Quantum-chemical representations may include information regarding, for example, energies or electronic properties of a compound.

### BRIEF DESCRIPTION OF THE DRAWINGS

FIG. 1 is a graph illustrating predictions of binding interactions with increasing numbers of libraries.

FIG. 2 is a graph illustrating multiple runs of predictions over time as the predictive models were improved.

### DETAILED DESCRIPTION OF THE INVENTION

The present disclosure provides virtual screenings methods for identifying compounds useful as therapeutic agents and/or useful as starting points for optimization in the development of therapeutic agents. These methods utilize large data sets of experimental data derived using DNA-encoded libraries to produce high confidence predictions of binding interactions between candidate compounds and proteins of interest.

### Encoded Compounds

This invention features methods utilizing encoded chemical entities including a chemical entity, one or more tags, and a headpiece operatively associated with the first chemical entity and one or more tags. The chemical entities, headpieces, tags, linkages, and bifunctional spacers are further described below.

### Chemical Entities

The encoded compounds (e.g., small molecules) utilized in the methods of the invention can include one or more building blocks and optionally include one or more scaffolds.

The scaffold S can be a single atom or a molecular scaffold. Exemplary single atom scaffolds include a carbon atom, a boron atom, a nitrogen atom, or a phosphorus atom, etc. Exemplary polyatomic scaffolds include a cycloalkyl group, a cycloalkenyl group, a heterocycloalkyl group, a heterocycloalkenyl group, an aryl group, or a heteroaryl group. Particular embodiments of a heteroaryl scaffold include a triazine, such as 1,3,5-triazine, 1,2,3-triazine, or 1,2,4-triazine; a pyrimidine; a pyrazine; a pyridazine; a furan; a pyrrole; a pyrrolline; a pyrrolidine; an oxazole; a pyrazole; an isoxazole; a pyran; a pyridine; an indole; an indazole; or a purine.

The scaffold S can be operatively linked to the tag by any useful method. In one example, S is a triazine that is linked directly to the headpiece. To obtain this exemplary scaffold, trichlorotriazine (i.e., a chlorinated precursor of triazine having three chlorines) is reacted with a nucleophilic group of the headpiece. Using this method, S has three positions having chlorine that are available for substitution, where two positions are available diversity nodes and one position is attached to the headpiece. Next, building block Aₙ is added to a diversity node of the scaffold, and tag Aₙ encoding for building block Aₙ (“tag Aₙ”) is ligated to the headpiece, where these two steps can be performed in any order. Then, building block Bₙ is added to the remaining diversity node, and tag Bₙ encoding for building block Bₙ is ligated to the end of tag Aₙ. In another example, S is a triazine that is operatively linked to a tag, where trichlorotriazine is reacted with a nucleophilic group (e.g., an amino group) of a PEG, aliphatic, or aromatic linker of a tag. Building blocks and associated tags can be added, as described above.

In yet another example, S is a triazine that is operatively linked to building block Aₙ. To obtain this scaffold, building block Aₙ having two diversity nodes (e.g., an electrophilic group and a nucleophilic group, such as an Fmoc-amino acid) is reacted with the nucleophilic group of a linker (e.g., the terminal group of a PEG, aliphatic, or aromatic linker, which is attached to a headpiece). Then, trichlorotriazine is reacted with a nucleophilic group of building block Aₙ. Using this method, all three chlorine positions of S are used as diversity nodes for building blocks. As described herein, additional building blocks and tags can be added, and additional scaffolds Sₙ can be added.

Exemplary building block Aₙ's include, e.g., amino acids (e.g., alpha-, beta-, gamma-, delta-, and epsilon-amino acids, as well as derivatives of natural and unnatural amino acids), chemical-reactive reactants (e.g., azide or alkyne chains) with an amine, or a thiol reactant, or combinations thereof. The choice of building block Aₙ depends on, for example, the nature of the reactive group used in the linker, the nature of a scaffold moiety, and the solvent used for the chemical synthesis.

Exemplary building block Bₙ's and Cₙ's include any useful structural unit of a chemical entity, such as optionally substituted aromatic groups (e.g., optionally substituted phenyl or benzyl), optionally substituted heterocyclyl groups (e.g., optionally substituted quinolinyl, isoquinolinyl, indolyl, isoindolyl, azaindolyl, benzimidazolyl, azabenzimidazolyl, benzisoxazolyl, pyridinyl, piperidyl, or pyrrolidinyl), optionally substituted alkyl groups (e.g., optionally substituted linear or branched C₁₋₆ alkyl groups or optionally substituted C₁₋₆ aminoalkyl groups), or optionally substituted carbocyclyl groups (e.g., optionally substituted cyclopropyl, cyclohexyl, or cyclohexenyl). Particularly useful building block Bₙ's and Cₙ's include those with one or more reactive groups, such as an optionally substituted group (e.g., any described herein) having one or optional substituents that are reactive groups or can be chemically modified to form reactive groups. Exemplary reactive groups include one or more of amine (—NR₂, where each R is, independently, H or an optionally substituted C₁₋₆ alkyl), hydroxy, alkoxy (—OR, where R is an optionally substituted C₁₋₆ alkyl, such as methoxy), carboxy (—COOH), amide, or chemical-reactive substituents. A restriction site may be introduced, for example, in tag Bₙ or Cₙ, where a complex can be identified by performing PCR and restriction digest with one of the corresponding restriction enzymes.

### Headpiece

In an encoded chemical entity, the headpiece operatively links each chemical entity to its encoding oligonucleotide tag. Generally, the headpiece is a starting oligonucleotide having at least two functional groups that can be further derivatized, where the first functional group operatively links the first chemical entity (or a component thereof) to the headpiece and the second functional group operatively links one or more tags to the headpiece. A bifunctional spacer can optionally be used as a spacing moiety between the headpiece and a chemical entity.

The functional groups of the headpiece can be used to form a covalent bond with a component of a chemical entity and another covalent bond with a tag. The component can be any part of the small molecule, such as a scaffold having diversity nodes or a building block. Alternatively, the headpiece can be derivatized to provide a spacer (e.g., a spacing moiety separating the headpiece from the small molecule to be formed in the library) terminating in a functional group (e.g., a hydroxyl, amine, carboxyl, sulfhydryl, alkynyl, azido, or phosphate group), which is used to form the covalent linkage with a component of the chemical entity. The spacer can be attached to the 5′-terminus, at one of the internal positions, or to the 3′-terminus of the headpiece. When the spacer is attached to one of the internal positions, the spacer can be operatively linked to a derivatized base (e.g., the C5 position of uridine) or placed internally within the oligonucleotide using standard techniques known in the art. Exemplary spacers are described herein.

The headpiece can have any useful structure. The headpiece can be, e.g., 1 to 100 nucleotides in length, preferably 5 to 20 nucleotides in length, and most preferably 5 to 15 nucleotides in length. The headpiece can be single-stranded or double-stranded and can consist of natural or modified nucleotides, as described herein. For example, the chemical moiety can be operatively linked to the 3′-terminus or 5′-terminus of the headpiece. In particular embodiments, the headpiece includes a hairpin structure formed by complementary bases within the sequence. For example, the chemical moiety can be operatively linked to the internal position, the 3′-terminus, or the 5′-terminus of the headpiece.

Generally, the headpiece includes a non-self-complementary sequence on the 5′- or 3′-terminus that allows for binding an oligonucleotide tag by polymerization, enzymatic ligation, or chemical reaction. The headpiece can allow for ligation of oligonucleotide tags and optional purification and phosphorylation steps. After the addition of the last tag, an additional adapter sequence can be added to the 5′-terminus of the last tag. Exemplary adapter sequences include a primer-binding sequence or a sequence having a label (e.g., biotin). In cases where many building blocks and corresponding tags are used (e.g., 100), a mix-and-split strategy may be employed during the oligonucleotide synthesis step to create the necessary number of tags. Such mix-and-split strategies for DNA synthesis are known in the art. The resultant library members can be amplified by PCR following selection for binding entities versus a target(s) of interest.

The headpiece or the complex can optionally include one or more primer-binding sequences. For example, the headpiece has a sequence in the loop region of the hairpin that serves as a primer-binding region for amplification, where the primer-binding region has a higher melting temperature for its complementary primer (e.g., which can include flanking identifier regions) than for a sequence in the headpiece. In other embodiments, the complex includes two primer-binding sequences (e.g., to enable a PCR reaction) on either side of one or more tags that encode one or more building blocks. Alternatively, the headpiece may contain one primer-binding sequence on the 5′- or 3′-terminus. In other embodiments, the headpiece is a hairpin, and the loop region forms a primer-binding site or the primer-binding site is introduced through hybridization of an oligonucleotide to the headpiece on the 3′ side of the loop. A primer oligonucleotide, containing a region homologous to the 3′-terminus of the headpiece and carrying a primer-binding region on its 5′-terminus (e.g., to enable a PCR reaction) may be hybridized to the headpiece and may contain a tag that encodes a building block or the addition of a building block. The primer oligonucleotide may contain additional information, such as a region of randomized nucleotides, e.g., 2 to 16 nucleotides in length, which is included for bioinformatics analysis.

The headpiece can optionally include a hairpin structure, where this structure can be achieved by any useful method. For example, the headpiece can include complementary bases that form intermolecular base pairing partners, such as by Watson-Crick DNA base pairing (e.g., adenine-thymine and guanine-cytosine) and/or by wobble base pairing (e.g., guanine-uracil, inosine-uracil, inosine-adenine, and inosine-cytosine). In another example, the headpiece can include modified or substituted nucleotides that can form higher affinity duplex formations compared to unmodified nucleotides, such modified or substituted nucleotides being known in the art. In yet another example, the headpiece includes one or more cross-linked bases to form the hairpin structure. For example, bases within a single strand or bases in different double strands can be cross-linked, e.g., by using psoralen.

The headpiece or complex can optionally include one or more labels that allow for detection. For example, the headpiece, one or more oligonucleotide tags, and/or one or more primer sequences can include an isotope, a radioimaging agent, a marker, a tracer, a fluorescent label (e.g., rhodamine or fluorescein), a chemiluminescent label, a quantum dot, and a reporter molecule (e.g., biotin or a his-tag).

In other embodiments, the headpiece or tag may be modified to support solubility in semi-, reduced-, or non-aqueous (e.g., organic) conditions. Nucleotide bases of the headpiece or tag can be rendered more hydrophobic by modifying, for example, the C5 positions of T or C bases with aliphatic chains without significantly disrupting their ability to hydrogen bond to their complementary bases. Exemplary modified or substituted nucleotides are 5′-dimethoxytrityl-N4-diisobutylaminomethylidene-5-(1-propynyl)-2′-deoxycytidine, 3′-[(2-cyanoethyl)-(N,N-diisopropyl)]-phosphoramidite; 5′-dimethoxytrityl-5-(1-propynyl)-2′-deoxyuridine, 3′-[(2-cyanoethyl)-(N,N-diisopropyl)]-phosphoramidite; 5′-dimethoxytrityl-5-fluoro-2′-deoxyuridine, 3′-[(2-cyanoethyl)-(N,N-diisopropyl)]-phosphoramidite; and 5′-dimethoxytrityl-5-(pyren-1-yl-ethynyl)-2′-deoxyuridine, or 3′-[(2-cyanoethyl)-(N,N-diisopropyl)]-phosphoramidite.

In addition, the headpiece oligonucleotide can be interspersed with modifications that promote solubility in organic solvents. For example, azobenzene phosphoramidite can introduce a hydrophobic moiety into the headpiece design. Such insertions of hydrophobic amidites into the headpiece can occur anywhere in the molecule. However, the insertion cannot interfere with subsequent tagging using additional DNA tags during the library synthesis or ensuing PCR once a selection is complete or microarray analysis, if used for tag deconvolution. Such additions to the headpiece design described herein would render the headpiece soluble in, for example, 15%, 25%, 30%, 50%, 75%, 90%, 95%, 98%, 99%, or 100% organic solvent. Thus, addition of hydrophobic residues into the headpiece design allows for improved solubility in semi- or non-aqueous (e.g., organic) conditions, while rendering the headpiece competent for oligonucleotide tagging. Furthermore, DNA tags that are subsequently introduced into the library can also be modified at the C5 position of T or C bases such that they also render the library more hydrophobic and soluble in organic solvents for subsequent steps of library synthesis.

In particular embodiments, the headpiece and the first tag can be the same entity, i.e., a plurality of headpiece-tag entities can be constructed that all share common parts (e.g., a primer-binding region) and all differ in another part (e.g., encoding region). These may be utilized in the “split” step and pooled after the event they are encoding has occurred.

In particular embodiments, the headpiece can encode information, e.g., by including a sequence that encodes the first split(s) step or a sequence that encodes the identity of the library, such as by using a particular sequence related to a specific library.

### Oligonucleotide Tags

The oligonucleotide tags described herein (e.g., a tag or a portion of a headpiece or a portion of a tailpiece) can be used to encode any useful information, such as a molecule, a portion of a chemical entity, the addition of a component (e.g., a scaffold or a building block), a headpiece in the library, the identity of the library, the use of one or more library members (e.g., use of the members in an aliquot of a library), and/or the origin of a library member (e.g., by use of an origin sequence).

Any sequence in an oligonucleotide can be used to encode any information. Thus, one oligonucleotide sequence can serve more than one purpose, such as to encode two or more types of information or to provide a starting oligonucleotide that also encodes for one or more types of information. For example, the first tag can encode for the addition of a first building block, as well as for the identification of the library. In another example, a headpiece can be used to provide a starting oligonucleotide that operatively links a chemical entity to a tag, where the headpiece additionally includes a sequence that encodes for the identity of the library (i.e., the library-identifying sequence). Accordingly, any of the information described herein can be encoded in separate oligonucleotide tags or can be combined and encoded in the same oligonucleotide sequence (e.g., an oligonucleotide tag, such as a tag, or a headpiece).

A building block sequence encodes for the identity of a building block and/or the type of binding reaction conducted with a building block. This building block sequence is included in a tag, where the tag can optionally include one or more types of sequence described below (e.g., a library-identifying sequence, a use sequence, and/or an origin sequence).

A library-identifying sequence encodes for the identity of a particular library. In order to permit mixing of two or more libraries, a library member may contain one or more library-identifying sequences, such as in a library-identifying tag (i.e., an oligonucleotide including a library-identifying sequence), in a ligated tag, in a part of the headpiece sequence, or in a tailpiece sequence. These library-identifying sequences can be used to deduce encoding relationships, where the sequence of the tag is translated and correlated with chemical (synthesis) history information. Accordingly, these library-identifying sequences permit the mixing of two or more libraries together for selection, amplification, purification, sequencing, etc.

A use sequence encodes the history (i.e., use) of one or more library members in an individual aliquot of a library. For example, separate aliquots may be treated with different reaction conditions, building blocks, and/or selection steps. In particular, this sequence may be used to identify such aliquots and deduce their history (use) and thereby permit the mixing together of aliquots of the same library with different histories (uses) (e.g., distinct selection experiments) for the purposes of the mixing together of samples together for selection, amplification, purification, sequencing, etc. These use sequences can be included in a headpiece, a tailpiece, a tag, a use tag (i.e., an oligonucleotide including a use sequence), or any other tag described herein (e.g., a library-identifying tag or an origin tag).

An origin sequence is a degenerate (random, stochastically-generated) oligonucleotide sequence of any useful length (e.g., about six oligonucleotides) that encodes for the origin of the library member. This sequence serves to stochastically subdivide library members that are otherwise identical in all respects into entities distinguishable by sequence information, such that observations of amplification products derived from unique progenitor templates (e.g., selected library members) can be distinguished from observations of multiple amplification products derived from the same progenitor template (e.g., a selected library member). For example, after library formation and prior to the selection step, each library member can include a different origin sequence, such as in an origin tag. After selection, selected library members can be amplified to produce amplification products, and the portion of the library member expected to include the origin sequence (e.g., in the origin tag) can be observed and compared with the origin sequence in each of the other library members. As the origin sequences are degenerate, each amplification product of each library member should have a different origin sequence. However, an observation of the same origin sequence in the amplification product could indicate multiple amplicons derived from the same template molecule. When it is desired to determine the statistics and demographics of the population of encoding tags prior to amplification, as opposed to post-amplification, the origin tag may be used. These origin sequences can be included in a headpiece, a tailpiece, a tag, an origin tag (i.e., an oligonucleotide including an origin sequence), or any other tag described herein (e.g., a library-identifying tag or a use tag).

Any of the types of sequences described herein can be included in the headpiece. For example, the headpiece can include one or more of a building block sequence, a library-identifying sequence, a use sequence, or an origin sequence.

Any of these sequences described herein can be included in a tailpiece. For example, the tailpiece can include one or more of a library-identifying sequence, a use sequence, or an origin sequence.

Any of tags described herein can include a connector at or in proximity to the 5′- or 3′-terminus having a fixed sequence. Connectors facilitate the formation of linkages (e.g., chemical linkages) by providing a reactive group (e.g., a chemical-reactive group or a photo-reactive group) or by providing a site for an agent that allows for a linkage (e.g., an agent of an intercalating moiety or a reversible reactive group in the connector(s) or cross-linking oligonucleotide). Each 5′-connector may be the same or different, and each 3′-connector may be the same or different. In an exemplary, non-limiting complex having more than one tags, each tag can include a 5′-connector and a 3′-connector, where each 5′-connector has the same sequence and each 3′-connector has the same sequence (e.g., where the sequence of the 5′-connector can be the same or different from the sequence of the 3′-connector). The connector provides a sequence that can be used for one or more linkages. To allow for binding of a relay primer or for hybridizing a cross-linking oligonucleotide, the connector can include one or more functional groups allowing for a linkage (e.g., a linkage for which a polymerase has reduced ability to read or translocate through, such as a chemical linkage).

These sequences can include any modification described herein for oligonucleotides, such as one or more modifications that promote solubility in organic solvents (e.g., any described herein, such as for the headpiece), that provide an analog of the natural phosphodiester linkage (e.g., a phosphorothioate analog), or that provide one or more non-natural oligonucleotides (e.g., 2′-substituted nucleotides, such as 2′-O-methylated nucleotides and 2′-fluoro nucleotides, or any described herein).

These sequences can include any characteristics described herein for oligonucleotides. For example, these sequences can be included in tag that is less than 20 nucleotides (e.g., as described herein). In other examples, the tags including one or more of these sequences have about the same mass (e.g., each tag has a mass that is about +/−10% from the average mass between within a specific set of tags that encode a specific variable); lack a primer-binding (e.g., constant) region; lack a constant region; or have a constant region of reduced length (e.g., a length less than 30 nucleotides, less than 25 nucleotides, less than 20 nucleotides, less than 19 nucleotides, less than 18 nucleotides, less than 17 nucleotides, less than 16 nucleotides, less than 15 nucleotides, less than 14 nucleotides, less than 13 nucleotides, less than 12 nucleotides, less than 11 nucleotides, less than 10 nucleotides, less than 9 nucleotides, less than 8 nucleotides, or less than 7 nucleotides).

Sequencing strategies for libraries and oligonucleotides of this length may optionally include concatenation or catenation strategies to increase read fidelity or sequencing depth, respectively. In particular, the selection of encoded libraries that lack primer-binding regions has been described in the literature for SELEX, such as described in Jarosch et al., Nucleic Acids Res. 34: e86 (2006), which is incorporated herein by reference. For example, a library member can be modified (e.g., after a selection step) to include a first adapter sequence on the 5′-terminus of the complex and a second adapter sequence on the 3′-terminus of the complex, where the first sequence is substantially complementary to the second sequence and result in forming a duplex. To further improve yield, two fixed dangling nucleotides (e.g., CC) are added to the 5′-terminus.

### Linkages

The linkages of the invention are present between oligonucleotides that encode information (e.g., such as between the headpiece and a tag, between two tags, or between a tag and a tailpiece). Exemplary linkages include phosphodiesters, phosphonates, and phosphorothioates. In some embodiments, a polymerase has reduced ability to read or translocate through one or more linkages. In certain embodiments, chemical linkages include one or more of a chemical-reactive group such as a monophosphate and/or a hydroxyl group, a photo-reactive group, an intercalating moiety, a cross-linking oligonucleotide, or a reversible co-reactive group.

A linkage may be tested to determine whether a polymerase has reduced ability to read or translocate through that linkage. This ability can be tested by any useful method, such as liquid chromatography-mass spectrometry, RT-PCR analysis, sequence demographics, and/or PCR analysis. In some embodiments, chemical ligation includes the use of one or more chemical-reactive pairs to provide a linkage such as a monophosphate and a hydroxyl. As described herein, readable linkages may be synthesized by chemical ligation, for example, by reaction of a monophosphate, a monophosphotioate, or monophosphanate on a 5′- or 3′-terminus with a hydroxyl group on a 5′- or 3′-terminus in the presence of cyanoimidazole and a divalent metal source (e.g., ZnCl₂).

Other exemplary chemical-reactive pairs are a pair including an optionally substituted alkynyl group and an optionally substituted azido group to form a triazole via a Huisgen 1,3-dipolar cycloaddition reaction; an optionally substituted diene having a 4 π-electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and an optionally substituted dienophile or an optionally substituted heterodienophile having a 2 π-electron system (e.g., an optionally substituted alkenyl group or an optionally substituted alkynyl group) to form a cycloalkenyl via a Diels-Alder reaction; a nucleophile (e.g., an optionally substituted amine or an optionally substituted thiol) with a strained heterocyclyl electrophile (e.g., optionally substituted epoxide, aziridine, aziridinium ion, or episulfonium ion) to form a heteroalkyl via a ring opening reaction; a phosphorothioate group with an iodo group, such as in a splinted ligation of an oligonucleotide containing 5′-iodo dT with a 3′-phosphorothioate oligonucleotide; an optionally substituted amino group with an aldehyde group or a ketone group, such as a reaction of a 3′-aldehyde-modified oligonucleotide, which can optionally be obtained by oxidizing a commercially available 3′-glyceryl-modified oligonucleotide, with 5′-amino oligonucleotide (i.e., in a reductive amination reaction) or a 5′-hydrazido oligonucleotide; a pair of an optionally substituted amino group and a carboxylic acid group or a thiol group (e.g., with or without the use of succinimidyl trans-4-(maleimidylmethyl)cyclohexane-1-carboxylate (SMCC) or 1-ethyl-3-(3-dimethylaminopropyl)carbodiimide (EDAC); a pair of an optionally substituted hydrazine and an aldehyde or a ketone group; a pair of an optionally substituted hydroxylamine and an aldehyde or a ketone group; or a pair of a nucleophile and an optionally substituted alkyl halide.

Platinum complexes, alkylating agents, or furan-modified nucleotides can also be used as a chemical-reactive group to form inter- or intra-strand linkages. Such agents can be used between two oligonucleotides and can optionally be present in the cross-linking oligonucleotide.

Exemplary, non-limiting platinum complexes include cisplatin (cis-diamminedichloroplatinum (II), e.g., to form GG intra-strand linkages), transplatin (trans-diaminedichloroplatinum (II), e.g., to form GXG inter-strand linkages, where X can be any nucleotide), carboplatin, picolatin (ZD0473), ormaplatin, or oxaliplatin to form, e.g., GC, CG, AG, or GG linkages. Any of these linkages can be inter- or intra-strand linkages.

Exemplary, non-limiting alkylating agents include nitrogen mustard (mechlorethamine, e.g., to form GG linkages), chlorambucil, melphalan, cyclophosphamide, prodrug forms of cyclophosphamide (e.g., 4-hydroperoxycyclophosphamide and ifosfamide)), 1,3-bis(2-chloroethyl)-1-nitrosourea (BCNU, carmustine), an aziridine (e.g., mitomycin C, triethylenemelamine, or triethylenethiophosphoramide (thio-tepa) to form GG or AG linkages), hexamethylmelamine, an alkyl sulfonate (e.g., busulphan to form GG linkages), or a nitrosourea (e.g., 2-chloroethylnitrosourea to form GG or CG linkages, such as carmustine (BCNU), chlorozotocin, lomustine (CCNU), and semustine (methyl-CCNU)). Any of these linkages can be inter- or intra-strand linkages.

Furan-modified nucleotides can also be used to form linkages. Upon in situ oxidation (e.g., with N-bromosuccinimide (NBS)), the furan moiety forms a reactive oxo-enal derivative that reacts with a complementary base to form an inter-strand linkage. In some embodiments, the furan-modified nucleotides forms linkages with a complementary A or C nucleotide. Exemplary, non-limiting furan-modified nucleotides include any 2′-(furan-2-yl)propanoylamino-modified nucleotide; or an acyclic, modified nucleotides of 2-(furan-2-yl)ethyl glycol nucleic acid.

Photo-reactive groups can also be used as a reactive group. Exemplary, non-limiting photo-reactive groups include an intercalating moiety, a psoralen derivative (e.g., psoralen, HMT-psoralen, or 8-methoxypsoralen), an optionally substituted cyanovinylcarbazole group, an optionally substituted vinylcarbazole group, an optionally substituted cyanovinyl group, an optionally substituted acrylamide group, an optionally substituted diazirine group, an optionally substituted benzophenone (e.g., succinimidyl ester of 4-benzoylbenzoic acid or benzophenone isothiocyanate), an optionally substituted 5-(carboxy)vinyl-uridine group (e.g., 5-(carboxy)vinyl-2′-deoxyuridine), or an optionally substituted azide group (e.g., an aryl azide or a halogenated aryl azide, such as succinimidyl ester of 4-azido-2,3,5,6-tetrafluorobenzoic acid (ATFB)).

Intercalating moieties can also be used as a reactive group. Exemplary, non-limiting intercalating moieties include a psoralen derivative, an alkaloid derivative (e.g., berberine, palmatine, coralyne, sanguinarine (e.g., iminium or alkanolamine forms thereof), or aristololactam-β-D-glucoside), an ethidium cation (e.g., ethidium bromide), an acridine derivative (e.g., proflavine, acriflavine, or amsacrine), an anthracycline derivative (e.g., doxorubicin, epirubicin, daunorubicin (daunomycin), idarubicin, and aclarubicin), or thalidomide.

For a cross-linking oligonucleotide, any useful reactive group (e.g., described herein) can be used to form inter- or intra-strand linkages. Exemplary reactive groups include chemical-reactive group, a photo-reactive group, an intercalating moiety, and a reversible co-reactive group. Cross-linking agents for use with cross-linking oligonucleotides include, without limitation, alkylating agents (e.g., as described herein), cisplatin (cis-diamminedichloroplatinum(II)), trans-diaminedichloroplatinum(II), psoralen, HMT-psoralen, 8-methoxypsoralen, furan-modified nucleotides, 2-fluoro-deoxyinosine (2-F-dl), 5-bromo-deoxycytosine (5-Br-dC), 5-bromo deoxyuridine (5-Br-dU), 5-iodo-deoxycytosine (5-I-dC), 5-iodo-deoxyuridine (5-I-dU), succinimidyl trans-4-(maleimidylmethyl)cyclohexane-1-carboxylate, SMCC, EDAC, or succinimidyl acetylthioacetate (SATA).

Oligonucleotides can also be modified to contain thiol moieties that can be reacted with a variety of thiol reactive groups such as maleimides, halogens, and iodoacetamides and thus can be used for cross-linking two oligonucleotides. The thiol groups can be linked to the 5′- or the 3′-terminus of an oligonucleotide.

For inter-strand cross-linking between duplex oligonucleotides at a pyrimidine (e.g., thymidine) position, the intercalating, photo-reactive moiety psoralen can be chosen. Psoralen intercalates into the duplex and forms covalent inter-strand cross-links with pyrimidines, preferentially at 5′-TpA sites, upon irradiation with ultraviolet light (about 254 nm). The psoralen moiety can be covalently attached to a modified oligonucleotide (e.g., by an alkane chain, such as a C₁₋₁₀ alkyl, or a polyethylene glycol group, such as —(CH₂CH₂O)ₙCH₂CH₂—, where n is an integer from 1 to 50). Exemplary psoralen derivatives can also be used, where non-limiting derivatives include 4′-(hydroxyethoxymethy)-4,5′,8-trimethylpsoralen (HMT-psoralen) and 8-methoxypsoralen.

Various portions of the cross-linking oligonucleotide can be modified to introduce a linkage. For example, terminal phosphorothioates in oligonucleotides can also be used for linking two adjacent oligonucleotides. Halogenated uracils/cytosines can also be used as cross-linker modifications in the oligonucleotide. For example, 2-fluoro-deoxyinosine (2-F-dl) modified oligonucleotides can be reacted with disulfide-containing diamines or thiopropylamines to form disulfide linkages.

As described below, reversible co-reactive groups include those selected from a cyanovinylcarbazole group, a cyanovinyl group, an acrylamide group, a thiol group, or a sulfonylethyl thioethers. An optionally substituted cyanovinylcarbazole (CNV) group can also be used in oligonucleotides to cross-link to a pyrimidine base (e.g., cytosine, thymine, and uracil, as well as modified bases thereof) in complementary strands. CNV groups promote [2+2] cycloaddition with the adjacent pyrimidine base upon irradiation at 366 nm, which results in an inter-strand cross-link. Irradiation at 312 nm reverses the cross-link and thus provides a method for reversible cross-linking of oligonucleotide strands. A non-limiting CNV group is 3-cyanovinylcarbozaole, which can be included as a carboxyvinylcarbazole nucleotide (e.g., as 3-carboxyvinylcarbazole-1′β-deoxyriboside-5′-triphosphate).

The CNV group can be modified to replace the reactive cyano group with another reactive group to provide an optionally substituted vinylcarbazole group. Exemplary non-limiting reactive groups for a vinylcarbazole group include an amide group of —CONR_{N1}R_{N2}, where each R_{N1} and R_{N2} can be the same or different and is independently H and C₁₋₆ alkyl, e.g., —CONH₂; a carboxyl group of —CO₂H; or a C₂₋₇ alkoxycarbonyl group (e.g., methoxycarbonyl). Furthermore, the reactive group can be located on the alpha or beta carbon of the vinyl group. Exemplary vinylcarbazole groups include a cyanovinylcarbazole group, as described herein; an amidovinylcarbazole group (e.g., an amidovinylcarbazole nucleotide, such as 3-amidovinylcarbazole-1′β-deoxyriboside-5′-triphosphate); a carboxyvinylcarbazole group (e.g., a carboxyvinylcarbazole nucleotide, such as 3-carboxyvinylcarbazole-1′β-deoxyriboside-5′-triphosphate); and a C₂₋₇ alkoxycarbonylvinylcarbazole group (e.g., an alkoxycarbonylvinylcarbazole nucleotide, such as 3-methoxycarbonylvinylcarbazole-1′β-deoxyriboside-5′-triphosphate). Additional optionally substituted vinylcarbazole groups and nucleotides having such groups are provided in the chemical formulas of U.S. Pat. No. 7,972,792 and Yoshimura and Fujimoto, Org. Lett. 10:3227-3230 (2008), which are both hereby incorporated by reference in their entirety.

Other reversible reactive groups include a thiol group and another thiol group to form a disulfide, as well as a thiol group and a vinyl sulfone group to form a sulfonylethyl thioethers. Thiol-thiol groups can optionally include a linkage formed by a reaction with bis-((N-iodoacetyl)piperazinyl)sulfonerhodamine. Other reversible reactive groups (e.g., such as some photo-reactive groups) include optionally substituted benzophenone groups. A non-limiting example is benzophenone uracil (BPU), which can be used for site- and sequence-selective formation of an interstrand cross-link of BPU-containing oligonucleotide duplexes. This cross-link can be reversed upon heating, providing a method for the reversible cross-linking of two oligonucleotide strands.

In other embodiments, chemical ligation includes introducing an analog of the phosphodiester bond, e.g., for post-selection PCR analysis and sequencing. Exemplary analogs of a phosphodiester include a phosphorothioate linkage (e.g., as introduced by use of a phosphorothioate group and a leaving group, such as an iodo group), a phosphoramide linkage, or a phosphorodithioate linkage (e.g., as introduced by use of a phosphorodithioate group and a leaving group, such as an iodo group).

For any of the groups described herein (e.g., a chemical-reactive group, a photo-reactive group, an intercalating moiety, a cross-linking oligonucleotide, or a reversible co-reactive group), the group can be incorporated at or in proximity to the terminus of an oligonucleotide or between the 5′- and 3′-termini. Furthermore, one or more groups can be present in each oligonucleotide. When pairs of reactive groups are required, then oligonucleotides can be designed to facilitate a reaction between the pair of groups. In the non-limiting example of a cyanovinylcarbazole group that co-reacts with a pyrimidine base, the first oligonucleotide can be designed to include the cyanovinylcarbazole group at or in proximity to the 5′-terminus. In this example, a second oligonucleotide can be designed to be complementary to the first oligonucleotide and to include the co-reactive pyrimidine base at a position that aligns with the cyanovinylcarbazole group when the first and second oligonucleotide hybridizes. Any of the groups herein and any of the oligonucleotides having one or more groups can be designed to facilitate reaction between the groups to form one or more linkages.

### Bifunctional Spacers

The bifunctional spacer between the headpiece and a chemical entity can be varied to provide an appropriate spacing moiety and/or to increase the solubility of the headpiece in organic solvent. A wide variety of spacers are commercially available that can couple the headpiece with the small molecule library. The spacer typically consists of linear or branched chains and may include a C₁₋₁₀ alkyl, a heteroalkyl of 1 to 10 atoms, a C₂₋₁₀ alkenyl, a C₂₋₁₀ alkynyl, C₅₋₁₀ aryl, a cyclic or polycyclic system of 3 to 20 atoms, a phosphodiester, a peptide, an oligosaccharide, an oligonucleotide, an oligomer, a polymer, or a poly alkyl glycol (e.g., a poly ethylene glycol, such as —(CH₂CH₂O)ₙCH₂CH₂—, where n is an integer from 1 to 50), or combinations thereof.

The bifunctional spacer may provide an appropriate spacing moiety between the headpiece and a chemical entity of the library. In certain embodiments, the bifunctional spacer includes three parts. Part 1 may be a reactive group, which forms a covalent bond with DNA, such as, e.g., a carboxylic acid, preferably activated by a N-hydroxy succinimide (NHS) ester to react with an amino group on the DNA (e.g., amino-modified dT), an amidite to modify the 5′ or 3′-terminus of a single-stranded headpiece (achieved by means of standard oligonucleotide chemistry), chemical-reactive pairs (e.g., azido-alkyne cycloaddition in the presence of Cu(I) catalyst, or any described herein), or thiol reactive groups. Part 2 may also be a reactive group, which forms a covalent bond with the chemical entity, either building block Aₙ or a scaffold. Such a reactive group could be, e.g., an amine, a thiol, an azide, or an alkyne. Part 3 may be a chemically inert spacing moiety of variable length, introduced between Part 1 and 2. Such a spacing moiety can be a chain of ethylene glycol units (e.g., PEGs of different lengths), an alkane, an alkene, a polyene chain, or a peptide chain. The spacer can contain branches or inserts with hydrophobic moieties (such as, e.g., benzene rings) to improve solubility of the headpiece in organic solvents, as well as fluorescent moieties (e.g. fluorescein or Cy-3) used for library detection purposes. Hydrophobic residues in the headpiece design may be varied with the spacer design to facilitate library synthesis in organic solvents. For example, the headpiece and spacer combination is designed to have appropriate residues wherein the octanol:water coefficient (Pod) is from, e.g., 1.0 to 2.5. Spacers can be empirically selected for a given small molecule library design, such that the library can be synthesized in organic solvent, for example, in 15%, 25%, 30%, 50%, 75%, 90%, 95%, 98%, 99%, or 100% organic solvent. The spacer can be varied using model reactions prior to library synthesis to select the appropriate chain length that solubilizes the headpiece in an organic solvent. Exemplary spacers include those having increased alkyl chain length, increased poly ethylene glycol units, branched species with positive charges (to neutralize the negative phosphate charges on the headpiece), or increased amounts of hydrophobicity (for example, addition of benzene ring structures).

Examples of commercially available spacers include amino-carboxylic spacers, such as those being peptides (e.g., Z-Gly-Gly-Gly-Osu (N-alpha-benzyloxycarbonyl-(Glycine)₃-N-succinimidyl ester) or Z-Gly-Gly-Gly-Gly-Gly-Gly-Osu (N-alpha-benzyloxycarbonyl-(Glycine)₆-N-succinimidyl ester, SEQ ID NO: 1)), PEG (e.g., Fmoc-aminoPEG2000-NHS or amino-PEG (12-24)-NHS), or alkane acid chains (e.g., Boc-ε-aminocaproic acid-Osu); chemical-reactive pair spacers, such as those chemical-reactive pairs described herein in combination with a peptide moiety (e.g., azidohomoalanine-Gly-Gly-Gly-OSu (SEQ ID NO: 2) or propargylglycine-Gly-Gly-Gly-OSu (SEQ ID NO: 3)), PEG (e.g., azido-PEG-NHS), or an alkane acid chain moiety (e.g., 5-azidopentanoic acid, (S)-2-(azidomethyl)-1-Boc-pyrrolidine, 4-azidoaniline, or 4-azido-butan-1-oic acid N-hydroxysuccinimide ester); thiol-reactive spacers, such as those being PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithio]-propionamido)hexanoate)); and amidites for oligonucleotide synthesis, such as amino modifiers (e.g., 6-(trifluoroacetylamino)-hexyl-(2-cyanoethyl)-(N,N-diisopropyl)-phosphoramidite), thiol modifiers (e.g., S-trityl-6-mercaptohexyl-1-[(2-cyanoethyl)-(N,N-diisopropyl)]-phosphoramidite, or chemical-reactive pair modifiers (e.g., 6-hexyn-1-yl-(2-cyanoethyl)-(N,N-diisopropyl)-phosphoramidite, 3-dimethoxytrityloxy-2-(3-(3-propargyloxypropanamido)propanamido)propyl-1-O-succinoyl, long chain alkylamino CPG, or 4-azido-butan-1-oic acid N-hydroxysuccinimide ester)). Additional spacers are known in the art, and those that can be used during library synthesis include, but are not limited to, 5′-O-dimethoxytrityl-1′,2′-dideoxyribose-3′-[(2-cyanoethyl)-(N,N-diisopropyl)]-phosphoramidite; 9-O-dimethoxytrityl-triethylene glycol, 1-[(2-cyanoethyl)-(N,N-diisopropyl)]-phosphoramidite; 3-(4,4′-dimethoxytrityloxy)propyl-1-[(2-cyanoethyl)-(N,N-diisopropyl)]-phosphoramidite; and 18-O-dimethoxytrityl hexaethyleneglycol, 1-[(2-cyanoethyl)-(N,N-diisopropyl)]-phosphoramidite. Any of the spacers herein can be added in tandem to one another in different combinations to generate spacers of different desired lengths.

Spacers may also be branched, where branched spacers are well known in the art and examples can consist of symmetric or asymmetric doublers or a symmetric trebler. See, for example, Newcome et al., Dendritic Molecules: Concepts, Synthesis, Perspectives, VCH Publishers (1996); Boussif et al., Proc. Natl. Acad. Sci. USA 92:7297-7301 (1995); and Jansen et al., Science 266:1226 (1994).

### Methods for Determining the Nucleotide Sequence of a Complex

This invention features a methods which include determining the nucleotide sequence of a complex, such that encoding relationships may be established between the sequence of the assembled tag sequence and the structural units (or building blocks) of the chemical entity. In particular, the identity and/or history of a chemical entity can be inferred from the sequence of bases in the oligonucleotide. Using this method, a library including diverse chemical entities or members (e.g., small molecules or peptides) can be addressed with a particular tag sequence.

Any of the linkages described herein can be reversible or irreversible. Reversible linkages include photo-reactive linkages (e.g., a cyanovinylcarbozole group and thymidine) and redox linkages. Additional linkages are described herein.

In an alternative embodiment, an “unreadable” linkage can be enzymatically repaired in order to generate a readable or at least translocatable linkage. Enzymatic repair processes are well known to those skilled in the art and include, but are not limited to, pyrimidine (e.g., thymidine) dimer repair mechanisms (e.g., using a photolyase or a glycosylase (e.g., T4 pyrimidine dimer glycosylase (PDG))), base excision repair mechanisms (e.g., using a glycosylase, an apurinic/apyrimidinic (AP) endonuclease, a Flap endonuclease, or a poly ADP ribose polymerase (e.g., human apurinic/apyrimidinic (AP) endonuclease, APE 1; endonuclease III (Nth) protein; endonuclease IV; endonuclease V; formamidopyrimidine [fapy]-DNA glycosylase (Fpg); human 8-oxoguanine glycosylase 1 (a isoform) (hOGG1); human endonuclease VIII-like 1 (hNEIL1); uracil-DNA glycosylase (UDG); human single-strand selective monofunctional uracil DNA glycosylase (SMUG1); and human alkyladenine DNA glycosylase (hAAG)), which can be optionally combined with one or more endonucleases, DNA or RNA polymerases, and/or a ligases for the repair), methylation repair mechanisms (e.g., using a methyl guanine methyltransferase), AP repair mechanisms (e.g., using an apurinic/apyrimidinic (AP) endonuclease (e.g., APE 1; endonuclease III; endonuclease IV; endonuclease V; Fpg; hOGG1; and hNEIL1), which can be optionally combined with one or more endonucleases, DNA or RNA polymerases, and/or a ligases for the repair), nucleotide excision repair mechanisms (e.g., using excision repair cross-complementing proteins or excision nucleases, which can be optionally combined with one or more endonucleases, DNA or RNA polymerases, and/or a ligases for the repair), and mismatch repair mechanisms (e.g., using an endonuclease (e.g., T7 endonuclease I; MutS, MutH, and/or MutL), which can be optionally combined with one or more exonucleases, endonucleases, helicases, DNA or RNA polymerases, and/or ligases for the repair). Commercial enzyme mixtures are available to readily provide these kinds of repair mechanisms, e.g., PreCR® Repair Mix (New England Biolabs Inc., Ipswich Mass.), which includes Taq DNA Ligase, Endonuclease IV, Bst DNA Polymerase, Fpg, Uracil-DNA Glycosylase (UDG), T4 PDG (T4 Endonuclease V), and Endonuclease VIII.

Methods for Encoding Chemical Entities within a Library

The methods of the invention may utilize a library having a diverse number of chemical entities that are encoded by oligonucleotide tags. Examples of building blocks and encoding DNA tags are found in U.S. Patent Application Publication No. 2007/0224607, the building blocks and tags of which are hereby incorporated by reference.

Each chemical entity is formed from one or more building blocks and optionally a scaffold. The scaffold serves to provide one or more diversity nodes in a particular geometry (e.g., a triazine to provide three nodes spatially arranged around a heteroaryl ring or a linear geometry).

The building blocks and their encoding tags can be added directly or indirectly (e.g., via a spacer) to the headpiece to form a complex. When the headpiece includes a spacer, the building block or scaffold is added to the end of the spacer. When the spacer is absent, the building block can be added directly to the headpiece or the building block itself can include a spacer that reacts with a functional group of the headpiece. Exemplary spacers and headpieces are described herein.

The scaffold can be added in any useful way. For example, the scaffold can be added to the end of the spacer or the headpiece, and successive building blocks can be added to the available diversity nodes of the scaffold. In another example, building block Aₙ is first added to the spacer or the headpiece, and then the diversity node of scaffold S is reacted with a functional group in building block Aₙ. Oligonucleotide tags encoding a particular scaffold can optionally be added to the headpiece or the complex. For example, Sₙ is added to the complex in n reaction vessels, where n is an integer more than one, and tag Sₙ (i.e., tag S₁, S₂, . . . , Sₙ₋₁, Sₙ) is bound to the functional group of the complex.

Building blocks can be added in multiple, synthetic steps. For example, an aliquot of the headpiece, optionally having an attached spacer, is separated into n reaction vessels, where n is an integer of two or greater. In the first step, building block Aₙ is added to each n reaction vessel (i.e., building block A₁, A₂, . . . Aₙ₋₁, Aₙ is added to reaction vessel 1, 2, . . . n−1, n), where n is an integer and each building block Aₙ is unique. In the second step, scaffold S is added to each reaction vessel to form an Aₙ-S complex. Optionally, scaffold Sₙ can be added to each reaction vessel to from an Aₙ-Sₙ complex, where n is an integer of more than two, and each scaffold Sₙ can be unique. In the third step, building block Bₙ is to each n reaction vessel containing the Aₙ-S complex (i.e., building block B₁, B₂, . . . Bₙ₋₁, Bₙ is added to reaction vessel 1, 2, . . . n−1, n containing the A₁-S, A₂-S, . . . Aₙ-S complex), where each building block Bₙ is unique. In further steps, building block Cₙ can be added to each n reaction vessel containing the Bₙ-Aₙ-S complex (i.e., building block C₁, C₂, . . . Cₙ₋₁, Cₙ is added to reaction vessel 1, 2, . . . n−1, n containing the Bₙ-Aₙ-S complex), where each building block Cₙ is unique. The resulting library will have n³ number of complexes having n³ tags. In this manner, additional synthetic steps can be used to bind additional building blocks to further diversify the library.

After forming the library, the resultant complexes can optionally be purified and subjected to a polymerization or ligation reaction, e.g., to a tailpiece. This general strategy can be expanded to include additional diversity nodes and building blocks (e.g., D, E, F, etc.). For example, the first diversity node is reacted with building blocks and/or S and encoded by an oligonucleotide tag. Then, additional building blocks are reacted with the resultant complex, and the subsequent diversity node is derivatized by additional building blocks, which is encoded by the primer used for the polymerization or ligation reaction.

To form an encoded library, oligonucleotide tags are added to the complex after or before each synthetic step. For example, before or after the addition of building block Aₙ to each reaction vessel, tag Aₙ is bound to the functional group of the headpiece (i.e., tag A₁, Aₙ is added to reaction vessel 1, 2, . . . n−1, n containing the headpiece). Each tag Aₙ has a distinct sequence that correlates with each unique building block Aₙ, and determining the sequence of tag Aₙ provides the chemical structure of building block Aₙ. In this manner, additional tags are used to encode for additional building blocks or additional scaffolds.

Furthermore, the last tag added to the complex can either include a primer-binding sequence or provide a functional group to allow for binding (e.g., by ligation) of a primer-binding sequence. The primer-binding sequence can be used for amplifying and/or sequencing the oligonucleotides tags of the complex. Exemplary methods for amplifying and for sequencing include polymerase chain reaction (PCR), linear chain amplification (LCR), rolling circle amplification (RCA), or any other method known in the art to amplify or determine nucleic acid sequences.

Using these methods, large libraries can be formed having a large number of encoded chemical entities. For example, a headpiece is reacted with a spacer and building block Aₙ, which includes 1,000 different variants (i.e., n=1,000). For each building block Aₙ, a DNA tag Aₙ is ligated or primer extended to the headpiece. These reactions may be performed in a 1,000-well plate or 10×100 well plates. All reactions may be pooled, optionally purified, and split into a second set of plates. Next, the same procedure may be performed with building block Bₙ, which also include 1,000 different variants. A DNA tag Bₙ may be ligated to the Aₙ-headpiece complex, and all reactions may be pooled. The resultant library includes 1,000×1,000 combinations of Aₙ×Bₙ (i.e., 1,000,000 compounds) tagged by 1,000,000 different combinations of tags. The same approach may be extended to add building blocks Cₙ, Dₙ, Eₙ, etc. The generated library may then be used to identify compounds that bind to the target. The structure of the chemical entities that bind to the library can optionally be assessed by PCR and sequencing of the DNA tags to identify the compounds that were enriched.

This method can be modified to avoid tagging after the addition of each building block or to avoid pooling (or mixing). For example, the method can be modified by adding building block Aₙ to n reaction vessels, where n is an integer of more than one, and adding the identical building block B₁ to each reaction well. Here, B₁ is identical for each chemical entity, and, therefore, an oligonucleotide tag encoding this building block is not needed. After adding a building block, the complexes may be pooled or not pooled. For example, the library is not pooled following the final step of building block addition, and the pools are screened individually to identify compound(s) that bind to a target. To avoid pooling all of the reactions after synthesis, a binding assay e.g. ELISA, SPR, ITC, Tm shift, SEC or similar, for example, may be used to monitor binding on a sensor surface in high throughput format (e.g., 384 well plates and 1,536 well plates). For example, building block Aₙ may be encoded with DNA tag Aₙ, and building block Bₙ may be encoded by its position within the well plate. Candidate compounds can then be identified by using a binding assay (e.g., ELISA, SPR, ITC, Tm shift, SEC or similar) and by analyzing the Aₙ tags by sequencing, microarray analysis and/or restriction digest analysis. This analysis allows for the identification of combinations of building blocks Aₙ and Bₙ that produce the desired molecules.

The method of amplifying can optionally include forming a water-in-oil emulsion to create a plurality of aqueous microreactors. The reaction conditions (e.g., concentration of complex and size of microreactors) can be adjusted to provide, on average, a microreactor having at least one member of a library of compounds. Each microreactor can also contain the target, a single bead capable of binding to a complex or a portion of the complex (e.g., one or more tags) and/or binding the target, and an amplification reaction solution having one or more necessary reagents to perform nucleic acid amplification. After amplifying the tag in the microreactors, the amplified copies of the tag will bind to the beads in the microreactors, and the coated beads can be identified by any useful method.

Once the building blocks from the first library that bind to the target of interest have been identified, a second library may be prepared in an iterative fashion. For example, one or two additional nodes of diversity can be added, and the second library is created and sampled, as described herein. This process can be repeated as many times as necessary to create molecules with desired molecular and pharmaceutical properties.

Various ligation techniques can be used to add the scaffold, building blocks, spacers, linkages, and tags. Accordingly, any of the binding steps described herein can include any useful ligation technique or techniques. Exemplary ligation techniques include enzymatic ligation, such as use of one of more RNA ligases and/or DNA ligases, as described herein; and chemical ligation, such as use of chemical-reactive pairs, as described herein.

### Screening Methods

There are multiple established technical methods to determine binding of compounds to proteins, e.g., by determining a Kd. Methods for detecting or quantifying binding of a compound to a target protein include, for example, absorbance, fluorescence, Raman scattering, phosphorescence, luminescence, luciferase assays, and radioactivity. Exemplary techniques include Surface Plasmon Resonance (SPR) and Fluorescence Polarization (FP). SPR measures the change in refractivity of a metal surface when a compound binds to a protein that is immobilized on that metal surface while FP measures the change in tumbling rate for a compound when it is bound to a protein using loss-of-polarization of incident light. In some embodiments, these methods may be used to experimentally determine the binding of a candidate compound predicted using the methods of the invention to bind a target protein.

Alternatively, compounds that bind to target proteins can be identified using affinity-based methods. For example, target proteins with affinity tags (e.g., poly-His tags) can be pre-incubated with a saturating concentration of one or more candidate compounds. Subsequent affinity purification and compound identification (e.g., through the utilization of an identity tag) would allow for the identification of compounds which bind to the target protein.

### Target Proteins

A target protein (e.g., a eukaryotic target protein such as a mammalian target protein or a fungal target protein or a prokaryotic target protein such as a bacterial target protein) is a protein which mediates a disease condition or a symptom of a disease condition. As such, a desirable therapeutic effect can be achieved by modulating (inhibiting or increasing) its activity.

Target proteins can be naturally occurring, e.g., wild type. Alternatively, a target protein can vary from the wild type protein but still retain biological function, e.g., as an allelic variant, a splice mutant or a biologically active fragment.

In some embodiments, the target protein is an enzyme (e.g., a kinase). In some embodiments, a target protein is a transmembrane protein. In some embodiments, a target protein has a coiled coil structure. In certain embodiments, a target protein is one protein of a dimeric complex.

In some embodiments, the target protein is a GTPase such as DIRAS1, DIRAS2, DIRAS3, ERAS, GEM, HRAS, KRAS, MRAS, NKIRAS1, NKIRAS2, NRAS, RALA, RALB, RAP1A, RAP1B, RAP2A, RAP2B, RAP2C, RASD1, RASD2, RASL10A, RASL10B, RASL11A, RASL11B, RASL12, REM1, REM2, RERG, RERGL, RRAD, RRAS, RRAS2, RHOA, RHOB, RHOBTB1, RHOBTB2, RHOBTB3, RHOC, RHOD, RHOF, RHOG, RHOH, RHOJ, RHOQ, RHOU, RHOV, RND1, RND2, RND3, RAC1, RAC2, RAC3, CDC42, RAB1A, RAB1B, RAB2, RAB3A, RAB3B, RAB3C, RAB3D, RAB4A, RAB4B, RABSA, RABSB, RABSC, RAB6A, RAB6B, RAB6C, RAB7A, RAB7B, RAB7L1, RAB8A, RAB8B, RAB9, RAB9B, RABL2A, RABL2B, RABL4, RAB10, RAB11A, RAB11B, RAB12, RAB13, RAB14, RAB15, RAB17, RAB18, RAB19, RAB20, RAB21, RAB22A, RAB23, RAB24, RAB25, RAB26, RAB27A, RAB27B, RAB28, RAB2B, RAB30, RAB31, RAB32, RAB33A, RAB33B, RAB34, RAB35, RAB36, RAB37, RAB38, RAB39, RAB39B, RAB40A, RAB40AL, RAB40B, RAB40C, RAB41, RAB42, RAB43, RAP1A, RAP1B, RAP2A, RAP2B, RAP2C, ARF1, ARF3, ARF4, ARF5, ARF6, ARL1, ARL2, ARL3, ARL4, ARL5, ARLSC, ARL6, ARL7, ARL8, ARL9, ARL10A, ARL10B, ARL10C, ARL11, ARL13A, ARL13B, ARL14, ARL15, ARL16, ARL17, TRIM23, ARL4D, ARFRP1, ARL13B, RAN, RHEB, RHEBL1, RRAD, GEM, REM, REM2, RIT1, RIT2, RHOT1, or RHOT2. In some embodiments, the target protein is a GTPas activating protein such as NF1, IQGAP1, PLEXIN-B₁, RASAL1, RASAL2, ARHGAPS, ARHGAP8, ARHGAP12, ARHGAP22, ARHGAP25, BCR, DLC1, DLC2, DLC3, GRAF, RALBP1, RAP1GAP, SIPA1, TSC2, AGAP2, ASAP1, or ASAP3. In some embodiments, the target protein is a Guanine nucleotide-exchange factor such as CNRASGEF, RASGEF1A, RASGRF2, RASGRP1, RASGRP4, SOS1, RALGDS, RGL1, RGL2, RGR, ARHGEF10, ASEF/ARHGEF4, ASEF2, DBS, ECT2, GEF-H1, LARG, NET1, OBSCURIN, P-REX1, P-REX2, PDZ-RHOGEF, TEM4, TIAM1, TRIO, VAV1, VAV2, VAV3, DOCK1, DOCK2, DOCK3, DOCK4, DOCK8, DOCK10, C3G, BIG2/ARFGEF2, EFA6, FBX8, or GEP100. In certain embodiments, the target protein is a protein with a protein-protein interaction domain such as ARM; BAR; BEACH; BH; BIR; BRCT; BROMO; BTB; C1; C2; CARD; CC; CALM; CH; CHROMO; CUE; DEATH; DED; DEP; DH; EF-hand; EH; ENTH; EVH1; F-box; FERM; FF; FH2; FHA; FYVE; GAT; GEL; GLUE; GRAM; GRIP; GYF; HEAT; HECT; IQ; LRR; MBT; MH1; MH2; MIU; NZF; PAS; PB1; PDZ; PH; POLO-Box; PTB; PUF; PWWP; PX; RGS; RING; SAM; SC; SH2; SH3; SOCS; SPRY; START; SWIRM; TIR; TPR; TRAF; SNARE; TUBBY; TUDOR; UBA; UEV; UIM; VHL; VHS; WD40; WW; SH2; SH3; TRAF; Bromodomain; or TPR. In some embodiments, the target protein is a heat shock protein such as Hsp20, Hsp27, Hsp70, Hsp84, alpha B crystalline, TRAP-1, hsf1, or Hsp90. In certain embodiments, the target protein is an ion channel such as Cav2.2, Cav3.2, IKACh, Kv1.5, TRPA1, NAv1.7, Nav1.8, Nav1.9, P2X3, or P2X4. In some embodiments, the target protein is a coiled-coil protein such as geminin, SPAG4, VAV1, MAD1, ROCK1, RNF31, NEDP1, HCCM, EEA1, Vimentin, ATF4, Nemo, SNAP25, Syntaxin 1a, FYCO1, or CEP250. In certain embodiments, the target protein is a kinase such as ABL, ALK, AXL, BTK, EGFR, FMS, FAK, FGFR1, 2, 3, 4, FLT3, HER2/ErbB2, HER3/ErbB3, HER4/ErbB4, IGF1R, INSR, JAK1, JAK2, JAK3, KIT, MET, PDGFRA, PDGFRB, RET RON, ROR1, ROR2, ROS, SRC, SYK, TIE1, TIE2, TRKA, TRKB, KDR, AKT1, AKT2, AKT3, PDK1, PKC, RHO, ROCK1, RSK1, RKS2, RKS3, ATM, ATR, CDK1, CDK2, CDK3, CDK4, CDK5, CDK6, CDK7, CDK8, CDK9, CDK10, ERK1, ERK2, ERK3, ERK4, GSK3A, GSK3B, JNK1, JNK2, JNK3, AurA, ARuB, PLK1, PLK2, PLK3, PLK4, IKK, KIN1, cRaf, PKN3, c-Src, Fak, PyK2, or AMPK. In some embodiments, the target protein is a phosphatase such as WIP1, SHP2, SHP1, PRL-3, PTP1B, or STEP. In certain embodiments the target protein is a ubiquitin ligase such as BMI-1, MDM2, NEDD4-1, Beta-TRCP, SKP2, E6AP, or APC/C. In some embodiments, the target protein is a chromatin modifier/remodeler such as a chromatin modifier/remodeler encoded by the gene BRG1, BRM, ATRX, PRDM3, ASH1L, CBP, KAT6A, KAT6B, MLL, NSD1, SETD2, EP300, KAT2A, or CREBBP. In some embodiments, the target protein is a transcription factor such as a transcription factor encoded by the gene EHF, ELF1, ELF3, ELF4, ELF5, ELK1, ELK5, ELK4, ERF, ERG, ETS1, ETV1, ETV2, ETV3, ETV4, ETV5, ETV6, FEV, FLI1, GAVPA, SPDEF, SPI1, SPIC, SPIB, E2F1, E2F2, E2F3, E2F4, E2F7, E2F8, ARNTL, BHLHA15, BHLHB2, BHLBHB3, BHLHE22, BHLHE23, BHLHE41, CLOCK, FIGLA, HASS, HES7, HEY1, HEY2, ID4, MAX, MESP1, MLX, MLXIPL, MNT, MSC, MYF6, NEUROD2, NEUROG2, NHLH1, OLIG1, OLIG2, OLIG3, SREBF2, TCF3, TCF4, TFAP4, TFE3, TFEB, TFEC, USF1, ARF4, ATF7, BATF3, CEBPB, CEBPD, CEBPG, CREB3, CREB3L1, DBP, HLF, JDP2, MAFF, MAFG, MAFK, NRL, NFE2, NFIL3, TEF, XBP1, PROX1, TEAD1, TEAD3, TEAD4, ONECUT3, ALX3, ALX4, ARX, BARHL2, BARX, BSX, CART1, CDX1, CDX2, DLX1, DLX2, DLX3, DLX4, DLX5, DLX6, DMBX1, DPRX, DRGX, DUXA, EMX1, EMX2, EN1, EN2, ESX1, EVX1, EVX2, GBX1, GBX2, GSC, GSC2, GSX1, GSX2, HESX1, HMX1, HMX2, HMX3, HNF1A, HNF1B, HOMEZ, HOXA1, HOXA10, HOXA13, HOXA2, HOXAB13, HOXB2, HOXB3, HOXBS, HOXC10, HOXC11, HOXC12, HOXC13, HOXD11, HOXD12, HOXD13, HOXD8, IRX2, IRX5, ISL2, ISX, LBX2,LHX2, LHX6, LHX9, LMX1A, LMX1B, MEIS1, MEIS2, MEIS3, MEOX1, MEOX2, MIXL1, MNX1, MSX1, MSX2, NKX2-3, NKX2-8, NKX3-1, NKX3-2, NKX6-1, NKX6-2, NOTO, ONECUT1, ONECUT2, OTX1, OTX2, PDX1, PHOX2A, PHOX2B, PITX1, PITX3, PKNOX1, PROP1, PRRX1, PRRX2, RAX, RAXL1, RHOXF1, SHOX, SHOX2, TGIF1, TGIF2, TGIF2LX, UNCX, VAX1, VAX2, VENTX, VSX1, VSX2, CUX1, CUX2, POU1F1, POU2F1, POU2F2, POU2F3, POU3F1, POU3F2, POU3F3, POU3F4, POU4F1, POU4F2, POU4F3, POU5F1P1, POU6F2, RFX2, RFX3, RFX4, RFX5, TFAP2A, TFAP2B, TFAP2C, GRHL1, TFCP2, NFIA, NFIB, NFIX, GCM1, GCM2, HSF1, HSF2, HSF4, HSFY2, EBF1, IRF3, IRF4, IRF5, IRF7, IRF8, IRF9, MEF2A, MEF2B, MEF2D, SRF, NRF1, CPEB1, GMEB2, MYBL1, MYBL2, SMAD3, CENPB, PAX1, PAX2, PAX9, PAX3, PAX4, PAX5, PAX6, PAX7, BCL6B, EGR1, EGR2, EGR3, EGR4, GLIS1, GLIS2, GLI2, GLIS3, HIC2, HINFP1, KLF13, KLF14, KLF16, MTF1, PRDM1, PRDM4, SCRT1, SCRT2, SNAI2, SP1, SP3, SP4, SP8, YY1, YY2, ZBED1, ZBTB7A, ZBTB7B, ZBTB7C, ZIC1, ZIC3, ZIC4, ZNF143, ZNF232, ZNF238, ZNF282, ZNF306, ZNF410, ZNF435, ZBTB49, ZNF524, ZNF713, ZNF740, ZNF75A, ZNF784, ZSCAN4, CTCF, LEF1, SOX10, SOX14, SOX15, SOX18, SOX2, SOX21, SOX4, SOX7, SOX8, SOX9, SRY, TCF7L1, FOXO3, FOXB1, FOXC1, FOXC2, FOXD2, FOXD3, FOXG1, FOXI1, FOXJ2, FOXJ3, FOXK1, FOXL1, FOXO1, FOXO4, FOXO6, FOXP3, EOMES, MGA, NFATS, NFATC1, NFKB1, NFKB2, TP63, RUNX2, RUNX3, T, TBR1, TBX1, TBX15, TBX19, TBX2, TBX20, TBX21, TBX4, TBX5, AR, ESR1, ESRRA, ESRRB, ESRRG, HNF4A, NR2C2, NR2E1, NR2F1, NR2F6, NR3C1, NR3C2, NR4A2, RARA, RARB, RARG, RORA, RXRA, RXRB, RXRG, THRA, THRB, VDR, GATA3, GATA4, or GATA5; or C-myc, Max, Stat3, androgen receptor, C-Jun, C-Fox, N-Myc, L-Myc, MITF, Hif-1alpha, Hif-2alpha, Bcl6, E2F1, NF-kappaB, StatS, or ER(coact). In certain embodiments, the target protein is TrkA, P2Y14, mPEGS, ASK1, ALK, Bcl-2, BCL-XL, mSIN1, RORγt, IL17RA, eIF4E, TLR7R, PCSK9, IgE R, CD40, CD40L, Shn-3, TNFR1, TNFR2, IL31 RA, OSMR, IL12beta1,2, Tau, FASN, KCTD 6, KCTD 9, Raptor, Rictor, RALGAPA, RALGAPB, Annexin family members, BOOR, NCOR, beta catenin, AAC 11, PLD1, PLD2, Frizzled7, RaLP, MLL-1, Myb, Ezh2, RhoGD12, EGFR, CTLA4R, GCGC (coact), Adiponectin R2, GPR 81, IMPDH2, IL-4R, IL-13R, IL-1R, IL2-R, IL-6R, IL-22R, TNF-R, TLR4, Nrlp3, or OTR.

### Virtual Screening Methods

Data Collection and Generation of Statistics

In some embodiments, a step in the virtual screening methods of the invention involves the acquisition of data derived from a DNA-encoded library selection experiment (e.g., an affinity based experiment) against a target protein. The selection data is read out as DNA sequences which are then aggregated into statistical readouts, e.g., sequence counts. The aggregation into statistics is based on grouping common encoded compounds, e.g., the putative chemical structure encoded by the DNA (instance level) or a partial sub-structure of that encoded chemistry (mono-, di- or tri-synthon level). A determination of whether a compound or partial compound is binding to the target (a binder) is made using cutoff values for the sequencing derived statistics from one or more selection conditions. Millions to 10s (or even 100s) of millions of sequences are used per selection condition in order to collect significant statistics reflective of true underlying small molecule/protein binding.

Machine Learning

Machine learning methods are known in the art, e.g., non-limiting machine learning methods include Naïve Bayes, Random Forest, Decision Tree, Support Vector Machine, Neural Net, and Deep Learning.

In some embodiments, each data point from the data collection step is used for training of a machine learning algorithm. Each data point includes information derived from a molecular structure of the compound (complete or partial) from the DNA-encoded library and the associated statistics from one or more selection experiments. The structure is used to generate numeric inputs (calculated chemical properties, e.g., molecular weight, c Log P) and binary strings (e.g., chemical fingerprints that reflect atoms, groups of atoms, and connectivity within the structure). These molecule calculated readouts are used as the input columns for training of and prediction by a machine learning algorithm. In some embodiments, the model is constructed such that the only inputs required are those derived directly from the structure of the molecule. In some embodiments, any structure for which these fingerprints and properties can be calculated can generate a prediction.

In some embodiments, further structural derivatives of the compound (e.g. core analysis which removes side chains) may be used to produce further fingerprint and property calculations, or alternative structural fingerprints used for training and prediction.

In some embodiments, data derived from one or more DNA-encoded library selection(s) are used to assess whether a molecule is deemed to represent an example of a binder (positive), a non-binder (negative), or a non-specific binder (negative). While the assessment (positive or negative) is based on the encoded molecules' behavior in at least one DNA-encoded library selection, additional information from other sources could be used to assess positive and negative classifications used for training. Of additional note, the structure of molecules known to have been synthesized in the library, but not exhibiting any counts from the sequencing are considered as negative examples in the training. In some embodiments, positive controls are included in the data sets. For example, binding interaction data from compounds with known binding affinities (e.g., known inhibitors or natural ligands) to the target protein may be included.

In one embodiment, the assessment of binding for an input molecule is determined through the detection of a statistically significant enrichment (elevated sequence counts) in a selection containing the target protein. The enrichment in a control condition where the target protein is not included is also used to assess the specificity of the binding. This condition will generally include a resin used for capturing the protein during selection, but without the addition of the protein. Additional information may be used in determining that a particular molecule or partial molecule is labelled as a positive, e.g., enrichment or non-enrichment in additional conditions or when selected against related proteins. Information derived from selections against a number of non-target proteins may also be used, for example a count of the total number of proteins against which a given molecule or partial molecule has been shown to demonstrate enrichment in a selection. For example, the detection of enrichment of a given molecule against several additional targets in a database may lead to a negative designation due to lack of specificity.

### Molecular Representations

In some embodiments of the invention, molecular representations are used to generate estimated binding calculations. Molecular representations include, for example, topological representations, electrostatic representations, geometric representations, or quantum-chemical representations. Topological representations may be based on atoms, features or functional groups and their connectivity (e.g., fingerprints, connection tables, molecular connectivity, and/or molecular graph representations). Electrostatic representations include, for example, surface electronics. Geometric representations are, e.g., pharmacophores, pharmacophore fingerprints, shape-based fingerprints, and/or 3D molecular coordinates using atoms, features, or functional groups. In some embodiments, quantum-chemical representations are used. In some embodiments, electronic molecular representations are chemical fingerprints.

In some embodiments, a step in the virtual screening methods of the invention involves the generation of chemical fingerprints for both the compounds for which binding interaction data has been generated and candidate compounds. Chemical fingerprints maybe generated using any method known in the art, e.g., ECFP6, FCFP6, ECFP4, MACCS, or Morgan/Circular Fingerprints. The chemical fingerprints are then analyzed to identify patterns, e.g., identify structural features which increase or decrease binding to a target protein. The information generated from the chemical fingerprint comparison of large numbers of compounds, e.g., at least 250,000 molecules, can be used to increase the accuracy of the generated estimated binding interactions compared to chemical fingerprint comparison of smaller numbers of compounds, e.g., under 100,000 compounds. In some embodiments, the chemical fingerprints are used in this method as the primary information for machine learning.

For example, an example training set input of an 8-bit fingerprint may include:

| | Fingerprint bits | | | | | | | | Training column |
|---|---|---|---|---|---|---|---|---|---|
| ID | 1 | 2 | 3 | 4 | 5 | 6 | 7 | 8 | Binds protein? |
| Compound1 | 1 | 0 | 0 | 1 | 1 | 0 | 1 | 1 | T |
| Compound2 | 1 | 1 | 0 | 0 | 0 | 0 | 1 | 1 | F |
| Compound3 | 1 | 1 | 0 | 1 | 1 | 0 | 0 | 0 | F |
| Compound4 | 0 | 0 | 1 | 1 | 1 | 0 | 1 | 0 | F |
| Compound5 | 1 | 1 | 1 | 1 | 1 | 0 | 0 | 1 | T |
| Compound6 | 1 | 0 | 0 | 0 | 0 | 0 | 0 | 1 | F |

The fingerprint is a representation of the chemical entities. The machine learning proceeds by feeding the training rows, i.e., each compound with the columns, i.e., fingerprint bits plus a training column indicating that it is a positive or negative example.

The algorithms (RF, Naïve Bayes, deep learning, neural nets, etc.) operate by looking for patterns that are correlated with true or false designations. These patterns may involve one or more bits. They may be discovered by explicitly analyzing statistics (e.g. Naïve Bayes, Random Forest) or through empirical feedback from varying model parameters (e.g. Neural Network).

Another approach that may be used is to add calculated property columns (e.g., MW, c Log P, tPSA) in addition to the fingerprints. In this case, the machine learning algorithm can utilize these additional columns in its statistical analysis or its model parameter search. The use of properties in the analysis can improve the accuracy of predictions when compared to prediction performed without the use of properties.

The molecules that are subsequently predicted in this approach are represented in the exact same way as those being represented in the training set with the key difference that the training column seen above is now an unknown. The model generates a predicted value to be filled into the binding characterization column (e.g., a binding prediction column). In some embodiments, the column is Boolean (T/F), categorical (e.g., non-binder, competitive binder, non-competitive binder, non-competitive binder), or numeric (e.g., a score reflecting probability of binder).

| | Fingerprint bits | | | | | | | | Properties | | | |
|---|---|---|---|---|---|---|---|---|---|---|---|---|
| ID | 1 | 2 | 3 | 4 | 5 | 6 | 7 | 8 | MW | cLogP | tPSA | Binds protein? |
| Compound1 | 1 | 0 | 0 | 1 | 1 | 0 | 1 | 1 | 504 | 3.2 | 160 | T |
| Compound2 | 1 | 1 | 0 | 0 | 0 | 0 | 1 | 1 | 612 | 5.3 | 94 | F |
| Compound3 | 1 | 1 | 0 | 1 | 1 | 0 | 0 | 0 | 453 | 4.6 | 112 | F |
| Compound4 | 0 | 0 | 1 | 1 | 1 | 0 | 1 | 0 | 476 | 1.7 | 185 | F |
| Compound5 | 1 | 1 | 1 | 1 | 1 | 0 | 0 | 1 | 598 | 7.1 | 131 | T |
| Compound6 | 1 | 0 | 0 | 0 | 0 | 0 | 0 | 1 | 485 | 3.3 | 87 | F |

Molecules for prediction, including fingerprint columns only may be used with the model generated by the first example above

| | Fingerprint bits | | | | | | | | |
|---|---|---|---|---|---|---|---|---|---|
| ID | 1 | 2 | 3 | 4 | 5 | 6 | 7 | 8 | Binds protein? |
| Compound1 | 0 | 1 | 0 | 0 | 1 | 0 | 1 | 1 | ? |
| Compound2 | 1 | 0 | 1 | 1 | 0 | 0 | 0 | 1 | ? |
| Compound3 | 1 | 1 | 1 | 1 | 1 | 1 | 0 | 0 | ? |

Below is an example prediction with input information extended to include properties which may be used with the model created by the second example above.

| | Fingerprint bits | | | | | | | | Properties | | | |
|---|---|---|---|---|---|---|---|---|---|---|---|---|
| ID | 1 | 2 | 3 | 4 | 5 | 6 | 7 | 8 | MW | cLogP | tPSA | Binds protein? |
| Compound1 | 0 | 1 | 0 | 0 | 1 | 0 | 1 | 1 | 467 | 5.4 | 135 | ? |
| Compound2 | 1 | 0 | 1 | 1 | 0 | 0 | 0 | 1 | 534 | 1.5 | 173 | ? |
| Compound3 | 1 | 1 | 1 | 1 | 1 | 1 | 0 | 0 | 399 | 4.5 | 97 | ? |

Output

In some embodiments, models generated will either produce a binary score indicating that a candidate compound is positive or negative, or a probability score (e.g. from 0 to 1) indicating the model's assignment of likelihood that a candidate compound is positive or negative for activity/binding. This value can then be used to make a go/no go decision on a given molecule (binary case) or inform a prioritization of candidate compounds (probability score).

### Examples

### Example 1

Selection data for soluble epoxide hydrolase (sEH) derived from a set of libraries was used to train one of several machine learning models (Random Forest, Naïve Bayes, or Neural Network) and then used to predict the selection behavior of molecules from libraries that were not included in the training set against the same target. The libraries used in the training set included a linear peptide library with 25,844,065 compounds, a 3-cycle pyrazole library with 3,976,320 compounds, a 2-cycle pyridine library with 5,079,459 compounds, and a 4-cycle macrocycle library with 1,511,399,304 compounds. The libraries used in the prediction set included a 3-cycle linear peptide library with 221,580,000 compounds, a 3-cycle pyridine library with 285,917,292 compounds, and a 2-cycle benzimidazole library with 1,622,820 compounds.

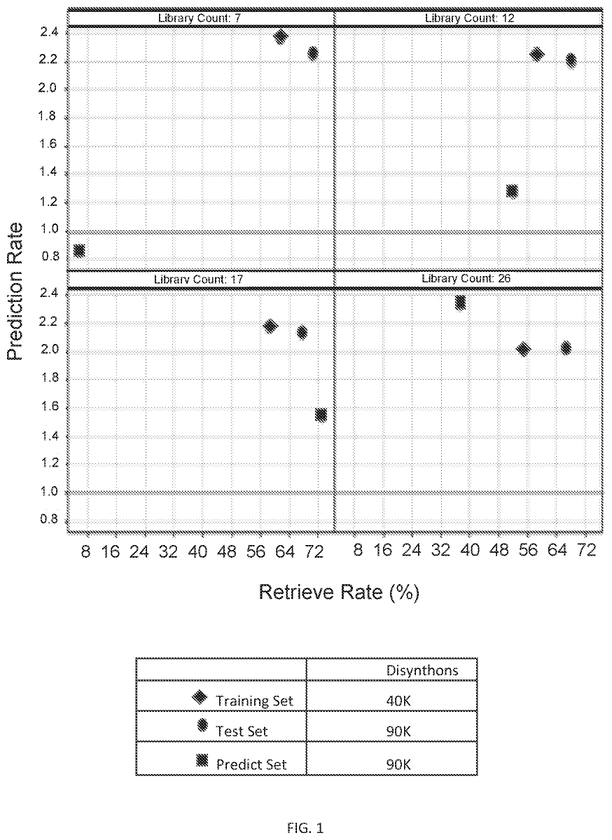

As shown in FIG. 1, enrichment of binders was seen in the predicted set. The 4 quadrants in the graph represent prediction of positive disynthons using increasing numbers libraries (left to right, top to bottom). The Y-axis represents the enrichment of positives in the predicted set as compared to random selection from the original population. The Y-axis shows the percentage of positives in the original set that were found in the predicted set. The result demonstrates that for the training and test sets (withheld disynthons not in the training set, but from the same libraries), the predicted set was consistently enriched 2-2.5 times the original population. The predict set are disynthons from the libraries not used in the training. In this case increasing the number of libraries used in training shows increasing rate of positives in the predicted population as compared to the original population.

### Example 2

Selection data from the same libraries as in Example 1 for a sEH was used with a machine learning algorithm (RF, MLP, deep learning) to train and produce a model that is used to predict activity of molecules not found in the DNA-encoded library. For example, data is fed in and a model is produced that can predict the activity of molecules tested in a traditional high throughput screening (HTS) experiment (i.e., robotic testing of 10 Ks to 1 Ms of molecules). The prediction by the model is applied as a filter to generate a list (e.g., 100s of compounds) from an initial list of 10,000 to 100,000 or more of molecules. The goal is to identify molecules in that short list such that the final list is vastly enriched (10× to 100×) over the underlying rate of active molecules found in the initial set.

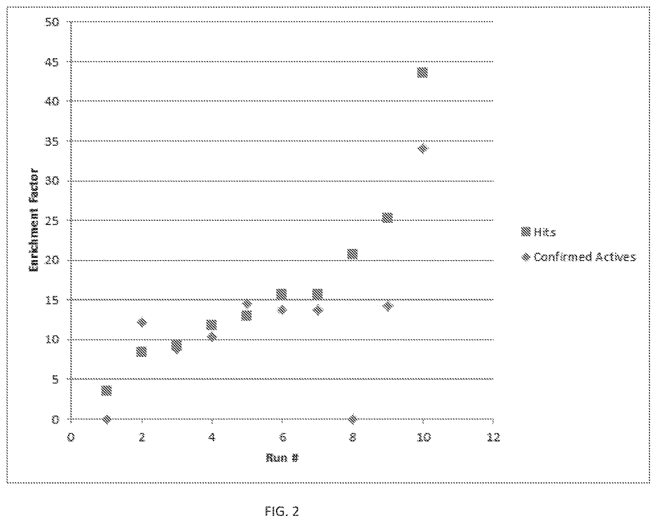

As shown in FIG. 2, enrichment of predicted molecules of >40× over random selection have been observed. FIG. 2 illustrates multiple runs over time as the predictive models were improved. The trend shows increasing enrichment of both primary HTS hits and the more stringent confirmed actives in the predicted set as compared to a random selection. The confirmed actives were subject to a secondary, confirmatory biochemical assay and demonstrated activity. The best result shows that the resulting predicted set has improved >40 times over random selection of molecules from the original population.

### Example 3. Optimization of Predictions

A known set of HTS data exists for a given target or targets. Multiple parameter settings are tested in order to achieve high prediction rates. In effect, the high prediction rate is a result of tuning to the prediction to the HTS results. Using HTS to confirm applicability, the model can then be used to predict novel compounds or existing compounds (e.g., commercially available or from a preexisting private compound library). These molecules can then be tested with the expectation of higher rate of actives, e.g., greater than 1% or 10% active molecules within the predicted set regardless of the underlying active rate of a random sample.

### Example 4. Optimization of Predictions

Data from selections against a given target, but under different conditions (e.g., using different protein fragments, mutants, isoforms, using closely related targets, using known small molecule competitors, etc.) are used to further refine the definition of positive data in the training set used to train the model.

### Example 5. Optimization of Predictions

Data from selections against 10s to 100s of protein targets, mutants, isoforms, etc. is used as a series of additional data columns in order to define a positive or negative example for training a machine learned model.

### OTHER EMBODIMENTS

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed:

1. A method comprising the steps of:
(a) providing a plurality of binding interaction findings for a target protein in a physical computing device having a representation of a set of candidate compounds,
wherein at least 90% of the binding interaction findings in the plurality are representative of a binding interaction between the target protein and a compound comprising a nucleotide tag encoding the identity of the compound;
(b) using the computing device to generate estimated binding interactions of the candidate compounds using the plurality of binding interaction findings; and
(c) outputting a candidate compound list capable of being displayed and ranked by highest estimated binding interactions.

2. The method of claim 1, wherein the plurality of binding interaction findings comprises at least one million binding interaction findings.

3. The method of claim 1, wherein at least 95% of the binding interaction findings in the plurality are representative of a binding interaction between the target protein and a compound comprising a nucleotide tag encoding the identity of the compound.

4. The method of claim 1, wherein at least 99% of the binding interaction findings in the plurality are representative of a binding interaction between the target protein and a compound comprising a nucleotide tag encoding the identity of the compound.

5. The method of claim 1, wherein at least 50% of the plurality of binding interaction findings were determined by contacting a plurality of compounds comprising a nucleotide tag encoding the identity of the compound with a target protein simultaneously.

6. The method of claim 1, wherein the method further comprises providing one or more additional pluralities of binding interaction findings for one or more additional target proteins, wherein at least 50% of the binding interaction findings in the plurality are representative of a binding interaction between the additional target protein and a compound from the plurality of binding interactions with the target protein.

7. The method of claim 6, wherein the candidate compound list is capable of being displayed and ranked by the selectivity of the candidate compound for the target protein over the one or more additional target proteins.

8. The method of claim 6, wherein the one or more additional target proteins comprise a mutant of the target protein.

9. The method of claim 1, wherein the method further comprises providing one or more additional pluralities of binding interaction findings for one or more negative control experiments, wherein at least 50% of the binding interaction findings in the plurality are representative of a negative control experiment of a compound from the plurality of binding interactions with the target protein.

10. The method of claim 1, wherein the method further comprises transmitting the candidate compound list over the internet or to a display device.

11. The method of claim 1, wherein the physical computing device is accessed and operated over the internet.

12. The method of claim 1, wherein the estimated binding interactions are generated using chemical structure comparisons.

13. The method of claim 12, wherein the chemical structure comparison utilizes molecular representations.

14. The method of claim 13, wherein the molecular representations comprise chemical fingerprints.

15. The method of claim 14, wherein the chemical fingerprint analysis is FCFP6, FCFP6, FCFP4, MACCS, or Morgan/Circular Fingerprints.

16. The method of claim 1, wherein the method further comprises generating a believability score for each of the estimated binding interactions of the candidate compounds, wherein the believability score is generated using chemical structure comparisons of the candidate compound and one or more compounds from the plurality of binding interactions for the target protein.

17. The method of claim 16, wherein the chemical structural comparison is principal component analysis.

18. The method of claim 16, wherein the candidate compound list is capable of being displayed and ranked by the believability score of the estimated binding interaction for the candidate compound.

19. The method of claim 1, wherein the method further comprises providing a one or more property findings for the set of candidate compounds.

20. The method of claim 19, wherein the one or more property findings include molecular weight and/or clog P.

21. The method of claim 19, wherein the one or more property findings are utilized to generate the estimated binding interactions.

22. The method of claim 19, wherein the candidate compound list is capable of being displayed and ranked by the one or more property findings.

23. The method of claim 1, wherein the method further comprises (d) synthesizing one or more of the candidate compounds from the candidate compound list.

24. The method of claim 23, wherein the method further comprises contacting the one or more synthesized candidate compounds with the target protein to determine one or more experimental binding interactions.

25. A computer readable medium having stored thereon executable instructions for directing a physical computing device to implement a method comprising the steps of:
(a) providing a plurality of binding interaction findings for a target protein in a physical computing device having a representation of a set of candidate compounds,
wherein at least 90% of the binding interaction findings in the plurality are representative of a binding interaction between the target protein and a compound comprising a nucleotide tag encoding the identity of the compound;
(b) using the computing device to generate estimated binding interactions of the candidate compounds using the plurality of binding interaction findings; and
(c) outputting a candidate compound list capable of being displayed and ranked by highest estimated binding interactions.

26. A physical computing device having a representation of a set of candidate compounds and programmed with executable instructions for directing the device to implement a method comprising the steps of:
(a) providing a plurality of binding interaction findings for a target protein in a physical computing device having a representation of a set of candidate compounds,
wherein at least 90% of the binding interaction findings in the plurality are representative of a binding interaction between the target protein and a compound comprising a nucleotide tag encoding the identity of the compound;
(b) using the computing device to generate estimated binding interactions of the candidate compounds using the plurality of binding interaction findings; and
(c) outputting a candidate compound list capable of being displayed and ranked by highest estimated binding interactions.
